(19) United States
(12) Patent Application Publication
(10) Pub. No.: US 2016/0194424 A1
(43) Pub. Date: Jul. 7, 2016

(54) MONOMERS FOR USE IN A POLYMERIZABLE COMPOSITION AND HIGH REFRACTIVE INDEX POLYMER FOR OPTHALMIC APPLICATIONS

(71) Applicant: CONTAMAC LTD, Saffron Walden, Essex (GB)

(72) Inventors: Timothy Charles Higgs, Saffron Walden (GB); Richard Alexander Young, Saffron Walden (GB)

(73) Assignee: Contamac Limited, Saffron Walden (GB)

(21) Appl. No.: 14/912,103

(22) PCT Filed: Aug. 12, 2014
(86) PCT No.: PCT/GB2014/052455

§ 371 Date: Feb. 15, 2016

(30) Foreign Application Priority Data
Aug. 13, 2013 (GB) 1314455.5

(51) Int. Cl. C08F 220/18; G02B 1/04; C07C 69/618

(52) U.S. Cl. CPC C08F 220/18; C07C 69/618; G02B 1/041

(57) ABSTRACT

Provided are monomers of formula (I), polymerizable compositions comprising the monomer of formula (I) optionally together with other co-polymerizable monomers and polymers obtained or obtainable from the polymerizable compositions, where the monomer of formula (I) is represented thus: Wherein —R¹ is —H or alkyl; —Z— is -0-, —NH—, or —N(R)—, where —R is optionally substituted alkyl or aryl; -Ar1 and -Ar2 are each independently optionally substituted aryl; and —R² is —H, or optionally substituted alkyl or aryl.

### RELATED APPLICATION

The present case claims the benefit and priority of GB 1314455.5 filed on 13 Aug. 2013, the contents of which are hereby incorporated by reference in their entirety.

### TECHNICAL FIELD

This invention generally pertains to polymerisable monomers and compositions for use in the preparation of polymer compounds. The polymers are suitable for use as ophthalmic lenses.

### BACKGROUND

Contact and intraocular ophthalmic lenses are devices for correcting defective vision. In particular, it has become commonplace to replace cataractous lens with an intraocular lens (IOLs) using surgical procedures.

A typical surgical procedure for lens replacement involves emulsifying the eye internal lens with an ultrasonic hand-piece inserted through a corneal incision. The disintegrated lens is then aspirated from the eye through the same incision, and a “rolled” IOL is then implanted via an injector into the eye through the same incision. In order to reduce surgical trauma, it is advantageous to minimize the size of the incision. It is for this reason that foldable IOLs were developed which can be folded into a cylindrical shape for injection along the axis of the “rolled lens” cylinder through a corneal incision. Once inserted and positioned under the pupil of the eye, the lens is permitted to unfurl, generally retuning to its original shape. The IOL is held in place by means of two or more small struts called haptics.

A significant class of foldable IOLs is formed from flexible polymers which are capable of slowly unfolding at the temperature of the eye (i.e. about 37′C) into an appropriate lens shape.

Hydrophobic acrylic-based polymers have been used for forming flexible IOLs of this type, e.g. as disclosed by U.S. Pat. No. 5,674,960, U.S. Pat. No. 5,922,821 and WO 96/40303. Such polymers are pliable, and have relatively high refractive indices, which enable the fabrication of thinner IOLs without concomitantly sacrificing optical refractory power. The overall dioptric power of the IOL depends on both the shape of the optic portion of the lens and the refractive index of the material from which the lens has been made.

There are two conventional manufacturing methods for hydrophobic-acrylic based IOLs. The first, which is suited to high volume output, involves a one-step moulding process whereby the lens shape is pre-determined by the shape of the mould holding the polymerizable composition used to fabricate the polymer. The second process involves fashioning a cylindrical ‘blank’ of the lens polymer into the required form using a high resolution lathing system combined with the milling of the haptics for a one-piece IOL design, or alternatively the affixing of separately fabricated haptics for a three-piece IOL. Hydrophobic-acrylic polymers capable of yielding IOLs that are easily foldable at room temperature exhibit relatively low glass transition temperatures, T_{g} typically of less than 20° C. This therefore requires the use of specialist lens fabrication equipment, such as cryo-lathes and cryo-mills, to cool the material to below its Tg during machining. The tooling addresses a firm non-pliable surface which ensures a high degree of precision, resulting in excellent optical quality with tight lens-to-lens tolerances.

The glass transition temperatures, Tg, for the foldable hydrophobic-acrylic based polymers are generally slightly lower than room temperature (ca. 23° C.) so that they are easily deformable at this temperature without causing physical damage to the polymer, for example by inducing creep, stress or fissures. On immersion in an aqueous environment for a prolonged period of time (e.g. vitreous humour in the posterior cavity of the eye) such low Tg hydrophobic polymers can be prone to the development of small “glistening bodies” in the polymer matrix. This occurs where temperature variations in a hydrophobic polymer can induce spinodal decomposition: small amounts of water entrained within the polymer matrix can “condense” on cooling, forming small water-filled cavities. These so-called vacuoles have a considerably lower refractive index (n_{D}²⁰=1.333) than the surrounding hydrophobic polymer matrix (typical n_{D}²⁰≧1.48) and therefore act as light scattering loci and appear to “glisten”.

A number of strategies have been employed to try to prevent the development of glistening bodies in hydrophobic-acrylic based polymers. The presence of glistening bodies is conventionally considered undesirable for this class of IOLs. Strategies to minimize vacuole formation have mostly concentrated on the modulation of the hydrophobicity of the polymer matrix through the inclusion of hydrophilic components (monomers, cross-linkers) into the root formulation. Through this approach it is anticipated that the polymer matrix can more effectively accommodate water, thereby reducing the propensity for glistening body formation.

The progressive incorporation of hydrophilic monomer(s) in the hydrophobic base formulation would ultimately yield a hydrogel polymer with appreciable water content. To clarify the distinction between a hydrogel and a polymer matrix accommodating water, US 2001/0003162 states that acrylic materials that absorb 5 wt °/0 or less water at 37° C. are considered to be non-hydrogel acrylic materials.

For example, U.S. Pat. No. 6,852,793 introduces N,N′-dimethylacylamide, and U.S. Pat. No. 7,789,509, U.S. Pat. No. 5,693,095 and WO 2006/063994 introduce 2-hydroxyethyl methacrylate into predominantly hydrophobic acrylic polymer formulations to enhance the water compatibility of the polymer matrix. In this way glistening body formation may be impeded, although the maximum 5 wt % equilibrium water content (the “hydrogel threshold”) may be surpassed.

An additional advantage of increased water content is the plasticising effect of the imbibed water, which conveniently hardens the dehydrated polymer during difficult mechanical processing steps (such as lathing or milling). U.S. Pat. No. 7,790,825 employs a slightly different approach to enhance the water compatibility of a hydrophobic-acrylic polymer. A ‘matrix hydrophilic modulation’ approach is followed where non-polymerisable block co-polymer surfactants, such as the Pluronic (BASF) range of poloxamers, are added into the matrix.

Some of the physical properties of the polymer used to make the IOL are dependent on the chemical structure of the monomer. For hydrophobic polymers based on acrylate or methacrylate monomers, the chemical functional group attached to the oxygen atom of the acryl- or methacryl-ester unit can influence the polymer's physical characteristics. In particular, a chemical functional group, which is known to impart particular physical characteristics to the resulting polymer, is covalently attached to the ester unit of the monomer by a bridging group, such as an alkyl chain. For example, U.S. Pat. No. 5,290,892, U.S. Pat. No. 5,403,901, U.S. Pat. No. 5,674,960 and U.S. Pat. No. 5,861,031 all disclose the attachment of an aromatic ring to the terminus of the alkyl bridging chain in order to impart a higher refractive index onto the monomer and the polymer formed from it. Furthermore these documents also disclose the insertion of heteroatoms such as sulfur, nitrogen or oxygen between the bridging alkyl-chain and the aromatic ring. The presence of sulfur is said to impart additional hydrophobicity and higher refractive index onto the resultant monomer.

This heteroatom concept is further developed in WO 00/79312, which discloses several classes of acrylate or methacrylate monomers for use in the preparation of homopolymer or copolymer products for IOL implants. The monomers contain an aryl functional group attached to the ester by an alkyl chain bridge where the alkyl bridging group may optionally also contain one or more oxygen or sulfur heteroatoms. Where the alkyl-chain bridge comprises multiple heteroatoms, these heteroatoms are dispersed evenly along the alkyl-chain in a polyether or polythioether motif. Where the alkyl-chain bridge comprises a single heteroatom, this atom forms an interlink link between the aryl and alkyl groups. For example, where the heteroatom is an oxygen atom, the group is an arylalkylether motif. Copolymers containing phenylthioethyl acrylate (i.e. an acrylate with an arylthio-alkyl side chain) are also prepared and characterized.

EP 1,792,923 and WO 2007/094665 disclose acrylic monomers possessing heteroatom arylalkylether or arylalkylthioether motifs where the refractive index is further amplified through the incorporation of more than one (typically two) arylalkylether or arylalkylthioether arms onto the core acryl- or methacryl-ester polymerisable functionality. The synthesis of the dual arm arylalkylthioether monomer, 1,3-bis(phenylthio)propan-2-yl methacrylate is disclosed in EP 1,792,923 and WO 2007/094665, as well as its use in preparing high refractive index hydrophobic polymer compositions.

The present inventors have previously described in WO 2011/107728 the use of acrylic monomers for a polymerisable composition, where the monomer possesses two arylalkyl arms that are connected to a fulcrum carbon. Each arm must have at least two carbon atoms within it, and the exemplified monomer is 1,5-diphenylpentan-3-yl acrylate (DPPA). Such a monomer may be said to contain a di(aryl)ethylene dual-arm moiety.

US 2011/0313518 describes aryl-containing monomers. The monomers possess a single aryl-containing arm.

U.S. Pat. No. 8,362,177 describes monomers identical to those in US 2011/0313518 for use in the preparation of ophthalmic devices. These monomers may be used together with another monomer having two aryl groups, such as 4,4′-dimethoxybenzhydryl methacrylate. Here, each aryl group is connected directly to a fulcrum carbon atom.

U.S. Pat. No. 7,354,980 describes the use of aryl-containing monomers, such as aryl acrylates. The worked examples are limited to the use of 2-phenyl acrylate and benzyl acrylate, \NO 2010/113600 discloses a variety of (meth)acrylate compounds for use in the preparation of polymer lenses.

The present invention is based on the finding that hydrophobic acrylic-based polymers having improved properties can be obtained from a class of acrylate, alkylacrylate, acrylamide or alkylacrylamide based monomers containing a di(aryl)methylene dual-arm moiety with optional substituents on either or both of the aryl rings.

### SUMMARY OF THE INVENTION

The present invention provides monomers and polymerisable compositions for use in the preparation of polymers for use in ophthalmic products (e.g. phakic, aphakic and pseudo-phakic intraocular lenses). The monomers of the invention may be used to produce polymers having improved optical properties, such as increased refractive index, and/or improved physical characteristics, such as folding/folding capability to permit smaller incision size during cataract lens replacement surgery. The monomers are particularly well suited to the preparation of polymers by photo-polymerization. The polymers obtainable from the monomers are suitable for use in ophthalmic lenses.

In a general aspect the invention provides an acrylate-based monomer having two aryl-bearing arms. The present inventors have established that the length of the arms, where each arm may be regarded as an arylmethylene group, is an important contributor the beneficial effects that are provided to the polymer product.

Accordingly, in a first aspect of the present invention provides a monomer for a polymerisable composition, the monomer having the formula (I):

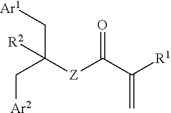

wherein:
—R¹ is —H or alkyl;
—Z— is —O—, —NH—, or —N(R)—, where —R is optionally substituted alkyl or aryl;
-Ar¹ and -Ar² are each independently optionally substituted aryl; and
—R² is —H, or optionally substituted alkyl or aryl.

In one embodiment, each of the phenyl rings present in the compound of formula (I) absorbs a negligible amount of electromagnetic radiation having a wavelength in the range 300-900 nm.

Where —R¹ is alkyl and —Z— is —O—, the monomer (I) may be referred to as an alkylacrylate monomer, for example where —R¹ is -Me, the monomer is a methacrylate. Where —R¹ is —H and —Z— is —O—, the monomer may be referred to as an acrylate monomer.

Where —R¹ is alkyl and —Z— is —NH— or —N(R)—, the monomer may be referred to as an alkylacrylamide monomer. Where —R¹ is —H and —Z— is —NH— or —N(R)—, the monomer may be referred to an as an acrylamide monomer.

In a second aspect of the invention there is provided a polymerisable composition comprising one or more monomers of formula (I).

In one embodiment, the polymerisable composition comprises further monomers, such as second, third and fourth monomers as described herein, for polymerization with the monomers of formula (I). In one embodiment, each of the further monomers has (alkyl)acrylate functionality, such as acrylate or methacrylate functionality. Here, the polymerisable composition may be referred to as a homo(alkyl)acrylate composition. In one embodiment, the composition is a homoacrylate composition.

In one embodiment, each second monomer, where present, has an aryl group, such as a phenyl group.

In the third aspect of the invention there is provided a polymer obtained or obtainable from a polymerisable composition comprising a monomer of formula (I).

In a fourth aspect of the invention there is provided a method for the synthesis of a polymer, the method comprising the step of polymerising a polymerisable composition comprising a monomer of formula (I).

In a fifth aspect of the invention there is provided a blank for an ophthalmic lens formed from the polymer of the third aspect of the invention.

In a sixth aspect of the invention there is provided an ophthalmic lens formed from the polymer of the third aspect of the invention.

In a seventh aspect of the invention there is provided a lens comprising a polymer of the third aspect of the invention, wherein the polymer is formed or formable in a lens-shaped mould.

In an eighth aspect of the invention there is provided a lens blank obtained or obtainable from a polymer of third aspect of the invention, wherein the lens blank has a fully formed optic zone and further comprising a zone, such as a ring, of polymer suitable for forming haptics portions.

In a ninth aspect of the invention there is provided a finished lens obtained or obtainable from the semi-finished blank of the eighth aspect of the invention.

Other aspects of the invention provide methods for the preparation of the blank of the fifth aspect of the invention and the ophthalmic lens of the sixth aspect of the invention.

The invention also provides the use of the polymer of the third aspect of the invention as an intraocular lens.

Further aspects and embodiments of the invention are as described below.

### DESCRIPTION OF THE FIGURE

FIG. 1 is a ¹H NMR spectrum of di(benzyl)methyl acrylate (DBMA) in CDCl₃ as prepared according to Example 2,

### DETAILED DESCRIPTION OF THE INVENTION

The present invention provides a monomer of formula (I) for use in a polymerisable composition. The monomer is provided with two aryl groups, individually attached to methylene spacers both of which are bound to a single fulcrum carbon atom, which is itself directly attached to the group, —Z—, which is the connection to polymerisable portion of the monomer. The monomer may be regarded as having two branches, each connecting the optionally substituted aryl groups to the polymerisable region of the monomer via a single fulcrum carbon atom.

A number of disclosures within the prior art describe monomer compounds having alkyl spacer groups that are interrupted with heteroatom functionality, such as sulfur or nitrogen atoms. Examples include monomers having single arms, such as described in WO 00/79312, U.S. Pat. No. 5,290,892, U.S. Pat. No. 5,403,901, U.S. Pat. No. 5,674,960, U.S. Pat. No. 5,861,031, and monomers having multiple arms, such as described in EP 1,792,923 and WO 2007/094665.

Many of the monomer compounds described in the prior art have a single alkyl arm linking the aryl functionality with the polymerisable region of the monomer, including, for example, U.S. Pat. No. 5,693,095, U.S. Pat. No. 6,780,899, U.S. Pat. No. 6,241,766, U.S. Pat. No. 6,271,281, U.S. Pat. No. 6,281,319, U.S. Pat. No. 6,326,448 and WO 00/79312. The present invention provides a monomer having two arms interlinking two phenyl functionalities as described above, and the present inventors have established that such monomers may be used to prepare polymers having a greater refractive index than is possible in those monomer compounds having a single alkyl arm linked to an aryl functionality (accordingly the monomer contains only a single aryl group).

Certain single- and multi-arm monomer compounds described in the prior art comprise aryl groups that are connected to an alkyl spacer group through electron-donating heteroatom functionality, such as sulfur and nitrogen. For such monomers the absorption characteristics of the aromatic conjugated phenyl chromophore are bathochromically shifted so that significant amounts of UVB (290-320 nm) and even UVC (320-400 nm) radiation are absorbed by this chromophore. In the absence of heteroatom substitution, an isolated non-fused phenyl ring would be expected to absorb predominantly UVC (100-290 nm) radiation.

The absorption of UVB and UVC radiation may compromise the long term stability of a polymer containing this functionality through photooxidative degradation phenomena. Conversely, solar UVC (100-290 nm) is not considered to pose a significant obstacle to achieving long-term polymer stability as it is almost entirely absorbed by stratospheric ozone, and the human cornea cuts off all light below wavelengths of 295 nm and only transmits approximately 1% of light at 300 nm.

The two methylene spacers present within each of the aryl-containing arms of the monomer (I) alleviate the steric hindrance of the polymerisable portion of the monomer. This results in excellent polymerisation conversion yields, especially under photo-polymerisation conditions. Furthermore the aryl-containing arms confer an attractive balance of properties for polymers containing a significant proportion of monomer(s) encompassed by formula (I) in that this moiety permits a degree of flexibility along the polymer backbone chain, through relaxation of the steric ring-chain interactions associated with (I), mediated by the methylene-spacers, whilst concomitantly imparting a relatively high glass transition temperature (Tg) onto the material.

The enhanced Tg is in part due to significant Van-Der-Waals attractions between the electron-rich aryl-rings of adjacent polymer chains containing units derived from monomer (I), possibly involving an aromatic-stacking mechanism. This observed Tg amplification for monomers encompassed by formula (I) is such that even many (alkyl)acrylate-forms of this system exhibit Tg values significantly above room temperature (23° C.) which is unusual for volatile (liquid) (alkyl)acrylate-based monomers due to the low steric congestion of the vinylic portion of the (alkyl)acrylate polymerisable group which yields very flexible, unconstrained, polymer chains. This Tg amplification is especially advantageous for acrylate-forms (where —Z— is —O—, and R¹ is —H) of formula (I) since the homopolymer Tg will be above room temperature but co-polymers incorporating (alkyl)acrylate-forms of monomer (I) together with co-monomers with Tg values below room temperature are not overly rigid due to the presence of the sterically relieving methylene aryl-acrylate interlinks between the phenyl and the polymerisable moiety which forms the polymer backbone chain.

When (alkyl)acrylate-forms of the formula (I) monomer are co-polymerised with lower Tg (alkyl)acrylate-based high refractive index monomer(s), such as 2-phenylethyl acrylate, this can yield a haze-free, low-tack polymer with a flexible “all acrylate” (homoacrylate) polymer-backbone with more flexural degrees-of-freedom than polymers containing one or more methacrylate monomers, which position sterically congesting methyl functionalities along the length of the polymer backbone chain. Homoacrylate polymerisable-compositions incorporating one or more acrylate-forms of the monomers encompassed by formula (I) together with any additional monomer components, including cross-linkers, also containing acrylate polymerisable functionalities yield polymers that exhibit advantageous unfolding properties expedient to a foldable small incision intraocular lens.

The inventors have discovered that diaryl-containing monomers of formula (I) are particularly useful for the preparation of short polymer chains during polymerization, for example during photo-polymerization. Under photo-polymerization conditions, complete monomer conversion occurs on a timescale of seconds to minutes rather than hours as is typically the case with thermally initiated conversion when employing a similar mole fraction of free-radical initiator in the polymerisable-composition. The former scenario necessarily results in much shorter polymer chains being formed under photo-polymerization conditions relative to the thermally polymerized situation, though the presence of cross-linking monomer(s) ensures that a “thermoset” type polymer is formed which is, for example, incapable of melt-flow deformation and furthermore is insoluble in all non-reactive solvents.

The difference in chain length for polymers produced using photo- and thermal-polymerisation techniques is well-recognised in the art. The respective time-scales for these modes of polymerisation, which contribute strongly to overall chain-length, are vastly different with shorter polymerisation times inevitably leading to shorter polymer chains. In the photo-polymerisation reaction, more radicals are produced in a set period, and as such will initiate more polymer chains and thus the available monomer(s) will be distributed between more chains leading to more, shorter chains.

Short polymer chains are inevitably less “tangled” with adjacent polymer chains and within the polymer there is a lower overall inter-chain Van-der-Waals attraction. The polymer therefore has greater mobility than an equivalent polymer-composition with longer polymer chains (where there are more entanglements and a higher number of Van-der-Waals attractive interactions will occur). It has been found that polymerisable-compositions containing a significant proportion (e.g. ≧25 wt %) of one or more monomers encompassed by formula (I) produce relatively short polymer chains, and yield materials that exhibit attractive mechanical characteristics including low tack and excellent folding/unfolding properties. Such benefits are particularly noticeable for the homoacrylate formulations discussed above.

It will be appreciated that relatively short polymer chains can be obtained by methods other than photo-polymerisation, for example, by employing elevated levels (>0.5 wt %) of a thermal free-radical initiator in a thermal polymerisation process, by thermal polymerisation at very elevated temperatures, or through the employment of a chain-transfer agent, such as α-methylstyrene dimer, n-butyl mercaptan, n-dodecyl mercaptan and the like, in a thermal free-radiated initiated polymer process.

The prior art describes dual- and multi-arm branched aryl acrylate monomers where each aryl ring is linked to a fulcrum carbon atom. It is the fulcrum carbon atom that is connected to the polymerisable acrylate moiety. See, for example, WO 2011/107728. The link between the aryl group and fulcrum carbon is a heteroatom-free alkylene linker. The alkylene linker has two or more carbon atoms e.g, ethylene, propylene etc. Conversely the prior art also describes multi-arm branched aryl acrylate monomers of a benzyhydryl-form where there is no linker between the aryl-ring and the fulcrum carbon. See, for example, U.S. Pat. No. 8,362,177. Neither WO 2011/107728 nor U.S. Pat. No. 8,362,177 describe a dual-arm branched aryl acrylate monomer where each aryl group is connected to the fulcrum carbon atom by a methylene (—CH₂—) linker.

Dual-arm branched aryl acrylate monomers having a heteroatom-free alkylene linking chains consisting of two or more carbon atoms have glass transition temperature (Tg) values too low to form viable high refractive index flexible polymers, and this is particularly the case where this is a significant component (for example, 25 wt %) of the polymerizable composition. Low polymer Tg values are also observed in homoacrylate polymerisable compositions of the type described above.

In contrast the use of monomers having no linker (benzyhydryl-forms) tends to result in polymers having Tg values that are too high. Moreover, these monomers impart considerable steric hindrance onto the resultant polymer chains, thereby reducing the flexural degrees-of-freedom. There is also a believed to be a reduction in the monomer conversion yield during the polymerization process. Even in homoacrylate polymerizable formulations, the resulting polymer is too rigid for satisfactory use, for example where the benzhydryl-containing monomer is a significant component (for example, ≧25 wt %) of the polymerizable composition. Here, the phenyl groups that are present in within each monomer will effectively crowd the polymer backbone to a greater degree than the aryl groups within the monomer of the present invention. This results in an increased Tg value.

Thus, the use of a monomer of formula (I) provides a polymer having useful Tg values and useful flexibility, and such advantages are available where the monomer is used at high quantities (for example, ≧25 wt %) with a polymerizable composition.

A further advantage of employing acrylate forms of monomers of formula (I) in homoacrylate systems, where all the monomer components possess polymerisable acrylate moieties, is the closer alignment of the rates of polymerisation of these monomers relative to mixed acrylate/methacrylate formulations (or those containing other combinations of polymerisable functionalities) which typically results in the formation of more homogeneous polymer matrices, with fewer and smaller domains of different refractive index which may mitigate issues pertaining to inter-domain matrix light-scattering which could deleteriously effect optical clarity (haze).

Acrylates are acknowledged to be significantly more reactive than methacrylate systems. In large part this is due to steric considerations, where the hindrance is caused by the methyl-substituent on the methacrylate-vinyl functionality. If acrylate and methacrylate monomers within a mixed polymerisable composition have significantly different refractive indices, then the differing reactivities will lead to large “homopolymer” domains of each of the monomer types (acrylate/methacrylate) and this will ultimately lead to light scattering as it passes through these domains and this is manifested as haze.

The nature and number of the aryl-ring substituents may be selected to enhance certain physical of the monomer. For example, the Tg of a polymer containing monomer(s) encompassed by formula (I) can be modulated through incorporation of alkyl- or alkoxy-ring substituents which will tend to decrease the polymer Tg. The hydrophobicity of the monomer can be increased by introducing fluorine containing substituents such as —F or —CF₃, as such are believed to inhibit water ingress into the polymer matrix. This is believed to be advantageous for it reduces the possibility that glistening bodies (microscopic vacuoles of water entrained within the polymer matrix induced by temperature fluctuations) will form in the polymer product.

The refractive index of the monomer (I) can be increased by introducing electron rich ring substituents, such as —Br atoms. The polymer prepared from such a monomer would be suitable for use in lenses with enhanced dioptric power, and therefore a constant lens shape. The degree of ring substitution can be used to further regulate the properties imparted by the ring substituent(s), for example, progressively increasing the degree of —Br aryl ring substitution will increase the refractive index of the resultant monomer and polymers derived therefrom.

In some embodiments, the compounds may have a chiral centre. For example, when the R² and R³ phenyl-ring substituents are different and/or m and n are different, the resulting monomer encompassed by formula (I) may be optically active. A chiral centre, or each chiral centre if more than one is present, is independently in the R- or the S-configuration. If no configuration is indicated, then both configurations are encompassed.

Polymerisable Composition

In a second aspect of the invention there is provided a polymerisable composition comprising one or more monomers of formula (I), optionally together with further monomers and other additives as described herein. The present inventors have established that polymer ophthalmic lenses, particularly intraocular lenses (IOLs), formed from such a composition may be suitably flexible to be folded or rolled to a size suitable for small incision surgical implantation. In further aspects of the invention there is provided a polymer, a lens and a lens blank obtained or obtainable from the polymerizable composition.

The polymerisable composition of the invention has a first monomer comprising one or more monomers of formula (I). The polymerisable composition may have from 5 to 99 wt % of the first monomer. The remaining portion of the polymerisable composition may comprise other monomer components and/or conventional polymerisation agents as described below.

In one embodiment, the first monomer is present in the composition in an amount of at least 5, 10, 15, or 20 wt %.

In one embodiment, the first monomer is present in the composition in an amount of at most 55, 65, 75, 85, 95 or 99 wt %.

In one embodiment, the first monomer is present in the composition in an amount selected from a range with the upper and lower amounts selected from the values given above. For example, the first monomer is present in an amount in the range 20 to 75 wt ° A, for example 20 to 65 wt %.

The polymerisable composition of the invention may further comprise one or more of a second monomer, one or more of a third monomer, and/or one or more of a fourth monomer, for copolymerisation with the first monomer. The second, third and/or fourth monomers may be used to adjust the physical and/or optical properties of the polymer product from the composition, as described below.

In one embodiment, each of the first and second, third and fourth monomers, where present, possess identical (alkyl)acrylate functionality, such as acrylate or methacrylate functionality. In an alternative embodiment, each of the first and second, third and fourth monomers, where present, possess (alkyl)acrylate functionality, and each monomer may have the same of different (alkyl)acrylate functionality. In each of these embodiments, the polymerizable composition may be referred to as a homo(alkyl)acrylate system. Thus, a (alkyl)homoacrylate system is a polymerisable composition where each monomer has an (alkyl)acrylate group. A homoacrylate system is a polymerisable composition where each monomer has an acrylate group.

In one embodiment, the second monomer is present in the composition in an amount of at least 5, 15, 20, 25, or 35 wt %.

In one embodiment, the second monomer is present in the composition in an amount of at most 55, 65, 75, or 85 wt %.

In one embodiment, the second monomer is present in the composition in an amount selected from a range with the upper and lower amounts selected from the values given above. For example, the second monomer is present in an amount in the range 15 to 75 wt %, for example 15 to 65 wt %.

In an alternative embodiment, the second monomer is present in an amount of at most 5, 10, 15, 20 or 25 wt %.

In one embodiment, the second monomer may be present in the polymerizable composition at a great or lesser amount than the first monomer (based on wt % or mole amount).

In one embodiment, the second monomer is a monomer having an acrylate or alkylacrylate, such as methacrylate, group for polymerization with the first monomer.

In one embodiment, the second monomer in the polymerizable composition has an aryl group, such as a phenyl group. Polymers formed from such compositions have improved optical and mechanical properties, such as lack of haze, good in hand foldability and good unfolding time. Examples of such monomers include di(phenylethyl)methyl acrylate and 2-phenylethyl acrylate.

Examples of second monomers include, but are not limited to, methyl acrylate, ethyl acrylate, n-propyl acrylate, n-butyl acrylate, t-butyl acrylate, n-hexyl acrylate, cyclohexyl acrylate, ethoxyethyl acrylate, methoxyethyl acrylate, methyl methacrylate, ethyl methacrylate, n-propyl methacrylate, n-butyl methacrylate, n-hexyl methacrylate, cyclohexyl methacrylate, ethoxyethyl methacrylate, methoxyethyl methacrylate, isobornyl methacrylate, isobornyl acrylate, 2-phenylethyl methacrylate, 2-phenylethyl acrylate, 1,4-diphenylbutan-2-yl acrylate, 1,4-diphenylbutan-2-yl methacrylate, 1,5-diphenylpentan-3-yl acrylate, 1,5-diphenylpentan-3-yl methacrylate and mixtures thereof.

In one embodiment, the second monomer is a monomer of formula (I) as described in the applicant's related published application, WO 2011/107728, the contents of which are hereby incorporated by reference in their entirety. In one embodiment, the second monomer is present in the polymerizable composition at in an amount of at most 25 wt %. In another embodiment, the second monomer is present in the polymerizable composition at in an amount from 15 to 75 wt % such as from 15 to 65 wt %.

In one embodiment, in addition or as an alternative to the second monomers mentioned above, the second monomer may be a monomer of formula (III):

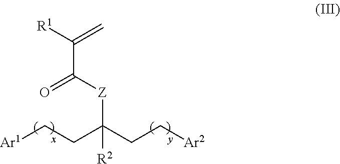

wherein:
—R¹ is —H or alkyl;
—Z— is —O—, —NH— or —NR—, where —R is optionally substituted alkyl or aryl;
-Ar¹ and -Ar² are each independently optionally substituted aryl;
—R² is —H, or optionally substituted alkyl or aryl; and
x and y are each independently 0 to 4, with the proviso that x and y are not both 0.

The groups —R¹, —Z—, —R², -Ar¹ and -Ar² in the second monomer of formula (III) may have the same meanings as —R¹, —Z—, —R², -Ar¹ and -Ar² in the monomer (I) of the present invention. The embodiments and preferences for R¹, —Z—, —R², -Ar¹ and -Ar² in the monomer (I) apply to the second monomers of formula (III).

Examples of second monomers of formula (III) include 1,4-diphenylbutan-2-yl acrylate, 1,4-diphenylbutan-2-yl methacrylate, 1,5-diphenylpentan-3-yl acrylate, and 1,5-diphenylpentan-3-yl.

In a further aspect of the invention there is provided a monomer of formula (III) as described above, wherein one of x and y is 0, and the other is 1. The invention also provides a polymerizable composition comprising the monomer of formula (III).

The second monomer may be selected so as to increase the tensile strength of the resulting polymer, for example by permitting the polymer to elongate a long way before breaking, or requiring a large load on the polymer (not necessarily contingent on having a high elongation before breaking) before it snaps.

In order to maintain a high overall refractive index whilst maintaining polymer flexibility, it is preferable to employ an acrylate monomer possessing an aromatic aryl group, such as 2-phenylethyl acrylate.

The third monomer is a crosslinking monomer. The crosslinking monomer is suitable for forming crosslinks with monomers in the polymerisable composition. Typically, the third monomer is provided with two or more reactive functional groups, such as olefinic groups, for reaction with suitable functionality on the first monomer, and/or the second monomer, and/or fourth monomer, where present. The third monomer may be provided with functional groups for cross-reactivity between third monomer molecules.

The polymerisable composition may have at least 0.2, 2, 5 or 15 wt of the third monomer.

In one embodiment, the third monomer is present in the composition in an amount of at least 0.1, 0.2, 0.5, 1, 2, 5, 10 or 15 wt %.

In one embodiment, the third monomer is present in the composition in an amount of at most 25, 50, 75, or 85 wt %.

In one embodiment, the third monomer is present in the composition in an amount selected from a range with the upper and lower amounts selected from the values given above. For example, the third monomer is present in an amount in the range 2 to 20 wt %.

Preferably the reactive functional groups of the third monomer are unsaturated functional groups such as double or triple bonds. The third monomer may be used to generate a three dimensional polymeric network in the polymerised product. The level of cross-linking monomer in the polymerisable composition may be adjusted to alter the material properties of the resulting polymer, most particularly the flexibility, optical clarity and elongation to break parameters.

Examples of third monomers include, but are not limited to polyethylene glycol dimethacrylate (PEG chain M_{w} 200-2,000), polyethylene glycol diacrylate (PEG chain M_{w} 200-2,000) polypropylene glycol dimethacrylate (PPG chain M_{w} 250-2,500), polypropylene glycol diacrylate (PPG chain M_{w} 250-2,500), ethylene glycol dimethacrylate, ethyleneglycol diacrylate, diethylene glycol dimethacrylate, diethylene glycol diacrylate, allyl acrylate, allyl methacrylate, 1,3-propanediol dimethacrylate, di-allyl maleate, 1,4-butanediol dimethacrylate and 1,4-butanediol diacrylate, 1,3-propanediol diacrylate, 1,3-propanediol dimethacrylate, 1,4-butanediol dimethacrylate, 1,6-hexanediol diacrylate, 1,6-hexanediol dimethacrylate, triethylene glycol diacrylate, triethylene glycol dimethacrylate, neopentyl glycol diacrylate, neopentyl glycol dimethacrylate, butylene glycol dimethacrylate, butylene glycol diacrylate, thio-diethylene glycol diacrylate, thio-diethylene glycol dimethacrylate, trimethylolpropane triacrylate, and diacrylates and dimethacrylates of bisphenol A, bisphenol A ethoxylate (1-3EO/phenol), bisphenol A propoxylate (1-3EO/phenol). Other crosslinking third monomers include N,N′-dihydroxyethylene bisacrylamide, diallyl phthalate, triallyl cyanurate, divinylbenzene, ethylene glycol divinyl ether. N,N-methylene-bis-(meth)acrylamide, sulfonated divinylbenzene, divinylsulfone and 1,3,5-triallyl-1,3,5-triazine-2,4,6(1H,3H,5H)-trione.

In order to maximize the degree-of-freedom and concurrent flexibility of the cross-linked polymer chain backbone it is preferable to employ an (alkyl)acrylate-based crosslinking monomer as the third monomer.

Furthermore it has been found that the employment of a polyethylene glycol chain interlinking the polymerisable moieties within the third monomer can serve a dual purpose, that of a functioning crosslinker whilst concomitantly having a plasticisation effect on the polymer so as to enhance its pliability and unfolding characteristics. The inventors have also found that the use of poly(oxyalklene) groups, such as polyethylene glycol, within the crosslinking monomer has the advantage of minimizing glistening body formation within the polymer product.

Thus, in one embodiment, the third monomer has a plurality of (alkyl)acrylate groups. In one embodiment, the third monomer has a plurality of (meth)acrylate groups, for example methacrylate and acrylate groups.

In one embodiment, the third monomer comprises a poly(oxyalklene) group, such as polyethylene glycol and polypropylene glycol.

The M_{W} of the third monomer may be at least 200, at least 500 or at least 1,000. The M_{W} of the third monomer may be at most 1,500, at most 2,000, or at most 5,000.

The preferred third monomer is polyethyleneglycol diacrylate where the polyethyleneglycol has a M_{w} in the range 700-1,000.

A fourth monomer may be present in the polymerisable composition. The fourth monomer is a hydrophilic monomer. The fourth monomer is suitable for polymerisation with the first monomer and additional monomers incorporated into the formulation.

In one embodiment, the fourth monomer is present in the composition in an amount of at least 0.1, 0.2, 0.5, 1, 2, 5, or 10 wt %.

In one embodiment, the fourth monomer is present in the composition in an amount of at most 15, 25, 40, or 50 wt %.

In one embodiment, the fourth monomer is present in the composition in an amount selected from a range with the upper and lower amounts selected from the values given above. For example, the fourth monomer is present in an amount in the range 0.1 to 50 wt %, such as 0.1 to 15 wt %.

The fourth hydrophilic monomer may be incorporated into the polymer to modulate, for example to increase or decrease the refractive index of the polymerised article and/or to control the mechanical properties of the polymer product through the plasticising effect of water. The inclusion of the hydrophilic monomer in the composition can also modulate the hydrophilicity of the polymer matrix, thereby reducing the propensity of the material to glistening body formation.

In one embodiment, the hydrophilic fourth monomers has a hydroxy group, such as hydroxyalkyl group, an amino group, including a carboxamide, or an alkoxy group, such as a poly(oxyalklene) group.

In one embodiment, the hydrophilic fourth monomer has an (alkyl)acrylate group for polymerization with the first monomer and other monomers, where present. In one embodiment, the fourth monomer has a (meth)acrylate group, for example a methacrylate or an acrylate groups.

Examples of hydrophilic fourth monomers include, but are not limited to, 2-hydroxyethyl methacrylate, 2-hydroxyethyl acrylate, 2-hydroxypropyl methacrylate, 2-hydroxypropyl acrylate, 3-hydroxypropyl methacrylate, 3-hydroxypropyl acrylate, 4-hydroxybutyl methacrylate, 4-hydroxybutyl acrylate, N-vinyl pyrrolidin-2-one, methacrylic acid, acrylic acid, acrylamide, methacrylamide, N,N′-dimethyl acrylamide, N-methyl-N-vinylacetamide, 2-hydroxy-3-phenoxypropyl acrylate, glycerol monomethacrylate, polyethylene glycol monomethacrylate (PEG chain M_{w}=200-2,000), polyethylene glycol methyl ether methacrylate (PEG chain M_{w}=200-2,000), polyethylene glycol monoacrylate (PEG chain M_{w}=200-2,000), polyethylene glycol methyl ether acrylate (PEG chain M_{w}=200-2,000) and N-(2-hydroxypropyl) methacrylamide and mixtures thereof.

The preferred fourth monomer is 2-hydroxyethyl acrylate.

It is noted that the second and third monomer may be hydrophilic or may include hydrophilic functionality. Such monomers may also modulate the refractive index and the hydrophilicity of the polymer product, as described above for the fourth monomer.

The polymerizable composition may further comprise conventional compounds for use on polymerization including, but not limited to, a thermally- or light-activated polymerisation initiator (preferably in an amount of up to 5 wt % of the composition), a “fixable”, for example by free-radical vinyl-polymerisation, UV-light absorber (also known as UV blockers, and are present preferably in an amount of up to 5 wt % of the composition), a “fixable” blue-light absorber (preferably in an amount of up to 0.5 wt % of the composition), a tackiness modifying agent, a strengthening agent, or a combination thereof. In one embodiment, the conventional compound comprises a functional group that is suitable for polymerisation with the first monomer and/or the second, third and fourth monomer where present.

As used herein, the term “fixable” is used in relation to a compound that may be incorporated into the polymer upon polymerisation of the polymerisable composition. Thus, a fixable compound is suitable for reaction with one or more of the first, second, third and fourth monomers, where present. Exemplary fixable monomers include those having vinyl functionalities for participation in, for instance, free-radical polymerisation with other vinyl-containing monomers, such as the first monomer described herein.

Examples of suitable UV-light absorbers include, but is not limited to, compounds including the benzoylphen-2-ol or 2-(2H-benzo[d][1,2,3]triazol-2-yl)phenol chromophore, such as 2-[3′-(2′H-benzotriazol-2′-yl)-4′-hydroxyphenyl]-ethylmethacrylate, 2-(4′-benzoyl-3′-hydroxyphenoxy)ethyl acrylate, 2-hydroxy-4-allyloxybenzophenone, 242′-hydroxy-5-methacryloxyethylphenyl)-2H-benzotriazole, β-(4-benzotriazoyl-3-hydroxyphenoxy)ethylacrylate, 4-(2-acryloxyethoxy)-2-hydroxybenzophenone, 4-methacryloyloxy-2-hydroxybenzophenone, 2-(2′-methacryloyloxy-5′-methylphenyl)benzotriazole, 2-(2′-hydroxy-5′-methacryloxyethylphenyl)-2H-benzotriazole, 2-[3′-tert-butyl-2′-hydroxy-5′-(3″-methacryloyloxypropyl)phenyl]-5-chlorobenzotriazole, 2-(3′-tert-butyl-5′-(3″-dimethylvinylsilylpropoxy)-2′-hydroxyphenyl]-5-methoxybenzotriazole, 2-(3′-allyl-2′-hydroxy-5′-methylphenyl) benzotriazole, 2-[3′-tert-butyl-2′-hydroxy-5′-(3″-methacryloyloxypropoxy)phenyl]-5-methoxybenzotriazole, 2-[3′-tert-butyl-2′-hydroxy-5′-(3″-methacyloyloxypropoxy)phenyl]-5-chlorobenzotriazole, 2-(2′-hydroxy-g-methacryloyloxyethylphenyl)-2H-benzotriazole and 2-(2′-hydroxy-3′-methallyl-5′-methylphenyl)benzotriazole. A preferred monomer as a UV-light absorber is 2-[3′-(2′H-benzotriazol-2′-yl)-4′-hydroxyphenyl]-ethylmethacrylate.

UV-light absorbers, such as those described herein, are known in the art to be exceptionally stable to both UVA and UVB solar radiation. The molecules are capable of absorbing light at wavelengths in these spectral ranges, and then dissipating this energy as heat. This dissipation occurs without the induction of potentially deleterious chemical reactivity, such as photooxidation, that could damage the integrity of the polymer. The incorporation of UV-blocking monomers into a polymerisable composition can therefore greatly extend the lifetime of a polymer subjected to solar radiation whilst concomitantly also having the beneficial effect of preventing UVB and UVC exposure to the ocular environment posterior to the IOL including the retina.

One or more tackiness modifying agents may be added to the polymerisable composition according to the present invention. The inclusion of a tackiness modifying component can advantageously yield a more tractable polymer product. A tackiness modifying agent may comprise at least one reactive unsaturated functional group, such as vinyl, acrylate or methacrylate-based groups.

Examples of tackiness modifying agents include, but is not limited to, fluorocarbon acrylates and methacrylates such as hexafluoro-iso-propyl methacrylate, 1H,1H,7H-dodecafluoroheptyl methacrylate, 1H,1H-heptafluorobutyl acrylate, 1H, 1H,3H-hexafluorobutyl methacrylate, 1H,1H,5H-octafluoropentyl methacrylate, 2,2,2-trifluoroethyl acrylate, and linear-chain alkyl acrylates or methacrylates such as butyl acrylate, butyl methacrylate, pentyl acrylate, pentyl methacrylate, hexyl acrylate, hexyl methacrylate, heptyl acrylate, heptyl methacrylate, octyl acrylate, octyl methacrylate and/or branched-chain alkyl acrylates or methacrylates such as isopentyl acrylate, isopentyl methacrylate, isobutyl acrylate, isobutyl methacrylate, 2,2-dimethylpropyl acrylate, 2,2-dimethyl propyl methacrylate, 2-ethylhexyl acrylate and 2-ethylhexyl methacrylate.

The polymerisable composition may comprise a thermally- or photo-activated polymerisation initiator. Preferably, the initiator is a free-radical polymerisation initiator.

The polymerisation initiator may be present in the polymerization composition in an amount of at most 0.5, 1.0, or 2.0 wt %

In a preferred embodiment, the polymerisable composition comprises 0.01 to 1.00 wt % of the polymerisation initiator.

Free-radical polymerisation of the polymerizable composition may be initiated thermally using a thermal free radical initiator such as peroxide, peroxidedicarbonate or azo-based initiators. Examples of peroxide or peroxidedicarbonate based initiators include, but are not limited to, dilauroyl peroxide, didecanoyl peroxide, tert-butyl peroxyneodecanoate, di(4-tert-butylcyclohexyl) peroxydicarbonate, dicetyl peroxydicarbonate, dimyristyl peroxydicarbonate. Examples of azo-based initiators include, but are not limited to, 1,1′-azobiscyanocyclohexane, 2,2′-azobis(2,4-dimethylvaleronitrile), 2,2′-azobisisobutyronitrile and 2,2′-azobis(2-methylbutyronitrile).

Photoactivated free-radical polymerisation of the polymerizable composition may be initiated by a photoinitiator, such as CIBA's Irgacure® 1800 [comprising 25% bis(2,6-dimethoxybenzoyl)-2,4,4-trimethyl-pentylphosphine oxide and 75% 1-hydroxy-cyclohexyl-phenyl ketone], Irgacure® 184 [comprising 100% 1-hydroxy-cyclohexyl-phenyl ketone], Irgacure® 819 [comprising 100% bis(2,4,6-trimethylbenzoyl)-phenylphosphine oxide], Irgacure® 2959 [comprising 100% 1-[4-(2-hydroxyethoxy)phenyl]-2-hydroxy-2-methyl-1-propane-1-one], Darocur® MBF [comprising 100% phenyl glyoxylic acid methyl ester], Darocur® TPO [comprising 100% 2,4,6-trimethylbenzoyl-diphenylphosphine oxide] and Darocur® 1173 [comprising 100% 2-hydroxy-2-methyl-1-phenyl-propan-1-one].

In embodiments where thermal polymerisation is employed in the poymerisation process, the preferred free-radical initiator is 2,2′-azobisisobutyronitrile (AIBN). Where photo-initiated free-radical polymerisation is employed to fabricate the hydrophobic-acrylic polymer composition, the preferred initiator bis(2,4,6-trimethylbenzoyl)-phenyl-phosphineoxide (for example, Irgacure 819).

The polymerisable composition may further comprise a diluent. The diluent may aid the processing of the polymer after polymerisation, particularly during the expulsion of extractable contaminants, such as residual monomers, by treatment with an appropriate solvent. A pre-swelled polymer network of the polymer having an incorporated diluent facilitates the removal of residual, leachable contaminants from the body of the polymer. Solvent extraction of a dry polymer typically causes swelling of the polymer body which can lead to a degradation of mechanical properties. This can be mitigated through the “pre-swelling” of the polymer network with a diluent at an appropriate level.

In one embodiment, the diluent is present in the composition in an amount of at least 1, 2, 5, or 10 wt % In one embodiment, the diluent is present in the composition in an amount of at most 15, 25, 30, 35, 40, or 50 wt %.

In one embodiment, the diluent is present in the composition in an amount selected from a range with the upper and lower amounts selected from the values given above. For example, the diluent is present in an amount in the range 2 to 40 wt %, such as 2 to 30 wt %.

Examples of suitable diluents include, but are not limited to, ethylene glycol, di(ethylene glycol), tetra(ethylene glycol), glycerol, 1,5-pentanediol, ethylene glycol monomethyl ether, ethylene glycol dimethyl ether, triethylene glycol monomethyl ether, 2-ethoxyethanol, solketal, benzonitrile, hexamethylphosphoramide, N-methylpyrrolidin-2-one and N,N′-dimethylformamide. Preferred diluents for inclusion in the polymerisable compositions of the present invention are N-methylpyrrolidin-2-one and N,N′-dimethylformamide.

It will be appreciated that the total amount of first monomer, second, third and fourth monomer, where present, conventional compounds, where present, and diluent, where present, does not exceed 100 wt %.

Polymers and Methods for their Preparation

In a third aspect of the invention there is provided a polymer obtained or obtainable from a polymerisable composition comprising a monomer of formula (I). The polymers are suitable for use in implantable medical devices, including ophthalmic devices such as IOLs.

The polymer of the invention is a polymer obtainable by polymerisation of a polymerisable composition of the invention. In one embodiment, the polymer is obtained or obtainable by free radical polymerisation of a polymerisable composition of the invention. In one embodiment, the polymer is obtained or obtainable by photo-initiated polymerization.

The polymer may be formed in a mould to provide a polymer product having a desired shape. For example, the polymer may be polymerized in a lens-shaped mould to yield a lens.

Alternatively, the polymer may be polymerized in a button or rod-shaped mould to yield a polymer button or rod. The button or rod may be subsequently machined to form a lens. Such methods are describe in further detail below.

The polymers of the invention comprise a unit of formula (II):

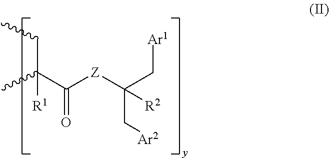

where —R¹, —Z—, —R², -Ar¹ and -Ar² are as defined for the monomers of formula (I), and y is the number of units.

In one embodiment, the polymer contains one or more units of formula (II). Thus y is one or more.

In one embodiment, the amount of unit (II) present in the polymer as a mole fraction of all the units present is at least 0.10, 0.15, 0.20, 0.30, 0.50, 0.70 or 0.80. The final mole fraction of (II) in the polymer may be altered by, for example, increasing or decreasing the amount of monomer of formula (I) in the polymerisable composition.

In one embodiment, the amount of unit (II) present in the polymer as a mole fraction of all the units present, is at most 0.85, 0.95 or 0.99.

The mole fraction may be determined from, for example, ¹H and/or ¹³C NMR measurements of the polymer product. Additionally or alternatively, the mole fraction may be surmised from the amount of first monomer in the polymerizable composition as a fraction of all the polymerizable monomers present.

In one embodiment, the number average of units of (I) present in a linear polymer-chain is at least 100, 500, 1,000, or 5,000. The chain length refers to the backbone length and does not refer to sections of polymer passing through branch points.

In one embodiment, the average M_{W} of a linear polymer-chain is at least 25,000 Da, or is at least 125,000 Da, or is least 250,000 Da, or is at least 1,250,000 Da. The chain length refers to the backbone length and does not refer to sections of polymer passing through branch points.

The M_{W} of a linear polymer-chain may be measured using standard techniques, for example from Tg measurements of the polymer, such as described by Thermal Analysis Consulting.

In one embodiment, the polymer has a Tg in the range of from −50 to 35° C., preferably from −20 to 30° C., or more preferably from −15 to 25° C. In one embodiment, the polymer has a Tg of less than 25° C.

Tg may be measured by dynamic mechanical thermal analysis (DMTA) as is well known to those skilled in the art. Exemplary methods and measurements are as described herein.

In one embodiment, the polymer has an elongation at 23° C. of at least 50%, at least 60%, at least 70%, or at least 75%.

In one embodiment, the polymer has an elongation at 23° C. of 50 to 300%, such as 100 to 300%, preferably 150 to 300%.

The elongation to break may be measured by tensile testing of a sample using a Zwick Z0.5 tensiometer, as is known to those skilled in the art.

In one embodiment, the polymer has a Tg of less than 25° C. and an elongation to break of at least 140%.

In one embodiment, the polymer has a refractive index at 20° C. of at least 1.50. It is preferred that the polymer has a refractive index of at least 1.50 and has an equilibrium water content in the range of 0 to 50 wt %.

The refractive index may be measured with an ABBE refractometer as is known to those skilled in the art.

### Ophthalmic Products and Methods for Manufacture

The invention also provides an ophthalmic lens comprising a polymer of the invention.

The ophthalmic lens of the invention is preferably an intraocular lens (104 Such lenses can either be described as phakic, aphakic or pseudophakic. A phakic lens is implantable in the eye without removal of the natural crystalline lens in a procedure to improve vision in patients with larger visual errors than typically seen in the general population. However an aphakic lens is implanted after removal of a clear crystalline lens with the goal again being an improvement in near or distance vision. Both these cases are examples of refractive surgery. A pseudophakic lens, the most common type of IOL, is used when the natural crystalline lens has been removed after developing a cataract. This procedure is the basis of cataract surgery. In addition to the types of lenses described, the placement of the lens within the eye is also used to describe the type of IOL implanted in patients. Such lenses are either implanted in the posterior segment of the eye, or the anterior segment of the eye.

Intraocular lenses may comprise optic and haptic portions. The optic portion comprises a mass of refracting material contained between two essentially spherical surfaces and is responsible for determining the visual functionality of the lens. The form of the optic portion (i.e. the curvature of its anterior and posterior surfaces), together with the refractive index of the polymer, determines the dioptric power of the lens. The haptic portion holds the lens in position beneath, and parallel to, the cornea after implantation and a key function of the haptic is to ensure the optic portion remains centred over the central visual zone of the eye. A single piece intraocular lens is manufactured from a single polymer blank and both the optic and haptic portions of the lens are usually formed simultaneously. A two or three piece IOL on the other hand usually comprises an optic portion manufactured from an individual polymer piece and the haptic portion or portions, which are produced from separate polymeric article(s), are subsequently attached to the optic portion in an additional manufacturing step.

The ophthalmic lens of the present invention may be described as having both an anterior surface and a posterior surface. In the case of an IOL, the posterior surface of the lens faces the back of the eye while the anterior surface is directed toward the front of the eye.

Further aspects of the present invention provide a blank for an ophthalmic lens formed from the polymer of the invention and an ophthalmic lens formed from the polymer of the invention.

The blank may be formed as a substantially cylindrical polymer product, with the cylinder typically having a circular diameter exceeding that of the altitude of the cylinder. The substantially cylindrical product may be formed from a cast moulding process using a suitable depression mould. The cylindrical polymer product may be worked, for example machined using milling and/or lathe cutting processes familiar to those skilled in the art, until a finished ophthalmic lens is obtained. The working process may also be referred to as machining of a shaped polymer product.

Alternatively, a mould may be used to fabricate a completely or substantially finished ophthalmic lens directly. Additional machining, typically involving the polishing of the optic portions of the lens, is usually required for a substantially finished ophthalmic lens to produce a useable lens.

The present invention also encompasses methods for fabricating a blank for an ophthalmic lens, and methods for fabricating an ophthalmic lens from a lens blank or from a polymer of a previous aspect of the invention.

A general method for fabricating an ophthalmic lens of the present invention comprises the steps of:
(a) providing a blank according to the present invention; and
(b) working the blank so as to form an ophthalmic lens.

Lens blanks according to the present invention may be manufactured according to any one of the methods described below. Reference to the shape or design of a mould as used herein refers to the shape or design of the part of the mould where the actual polymerisation of the polymer takes place.

A first method of forming a blank for an ophthalmic lens comprises the steps of:
(a) polymerisation of a composition of the present invention in a substantially rod-shaped mould thereby to form a polymer rod; and
(b) working the polymer rod into a plurality of cylindrical blanks.

A polymerisation reaction on a polymerisable composition of the present invention may be performed in the mould to form the polymer. Alternatively a preformed linear polymer may be placed in the mould and then cured to obtain the desired polymer product. An example of polymerisation in the mould is described below in the button moulding method.

A substantially rod-shaped (i.e. cylindrical) mould is typically constructed from polypolypropylene, polyethylene, PTFE or glass. The shape and size of the mould determines the diameter of the polymer rod. The diameter for the polymer rod is chosen for the design of the resulting ophthalmic lens to be formed; a larger diameter rod is required for a single piece ophthalmic lens and a smaller diameter rod is sufficient for a two or three-piece design ophthalmic lens. Typically, the polymer rod formed is worked into a series of homogeneous discs as described above. Generally the discs have parallel faces.

In an alternative method, a blank for an ophthalmic lens may be formed in a method comprising the step of polymerisation of a polymerisable composition according to the invention in a button mould thereby forming a lens blank. A polymerisation reaction on the polymerisable composition of the present invention may be performed in the button mould to form the polymer. An uncured polymer may be placed in the mould and cured, as an alternative to this method.

Typically, button moulds consist of an array of button impressions on a pre-formed polypropylene, polyethylene or PTFE sheet. The dimensions of the individual button moulds are determined by the resulting design of the final lens. Button moulds with a larger diameter button are required for a single piece ophthalmic lens, and a smaller diameter button mould is sufficient for a two or three-piece design ophthalmic lens.

The mould-sheet is covered with a lid-stock, typically comprising polyethylene or polypropylene. The lid-stock covered mould-sheet is filled with the polymer composition of the present invention and the mould is sealed, for example using a heat-sealing bar apparatus. A monomer formulation may be polymerised in the mould using an oven or, more preferentially, in a water bath thermally equilibrated to the required polymerisation temperature.

Once the polymerisation step has been completed, the water bath is allowed to cool and the mould-sheet is removed, cleaned and dried. The lid-stock can then be peeled from the mould and the polymerised discs extruded. It may be advantageous to perform the lid-stock removal and mould extrusion at a reduced temperature to prevent possible damage to the relatively soft polymer disc. This is particularly important when diluents are employed in the polymerisable composition. In such instances the mould can be chilled to a temperature lower than that of the freezing point of the diluent (or the Tg of the polymer where a diluent is not employed) for a period of 5 to 60 minutes immediately prior to lid-stock removal and subsequent mould extrusion.

Both of the above moulding methods for providing a lens blank may include an additional step of flushing the polymer rod initially formed after the polymerisation step. The flushing step comprises treating the polymer rods or buttons with an appropriate solvent or solvent mixture to remove extractable contaminants. An example of a suitable solvent for extracting contaminants is acetone and an example of a suitable solvent mixture for contaminant extraction is acetone/n-hexane.

It may also be desirable to include a drying step after the polymerisation step, and after any flushing step. The polymer rod or disc may be dried or annealed at an elevated temperature, either in air, an inert atmosphere of nitrogen or argon or under reduced pressure. The drying step may be carried out at a temperature in the range 30 to 150° C. Preferably, the drying or annealing step is performed under reduced pressure in the range 0.001 to 300 torr. Preferably the pressure is in the range 0.01 to 10 torr, most preferably 0.03 to 0.50 torr.

A lens blank obtained using the above moulding methods may be ground and polished such that the dimensions of the disc or blank lies within a stringent tolerance window with respect to the accuracy of both the diameter of the disc and the altitude between the opposing circular faces and their degree of parallelism.

The present invention also provides a method for preparing an ophthalmic lens, wherein a lens blank is lathe cut and optionally machine milled into a required lens shape. The step of machining a blank or polymer disc to form an ophthalmic lens comprises the following steps:
(a) lathe machining a first surface of an ophthalmic lens from a lens blank,
(b) lathe machining a second surface of an ophthalmic lens from the lens blank.

In some circumstances it may be preferable to first machine the anterior surface of the IOL followed by the posterior surface. Alternatively, the posterior surface may be machined first.

Before each lathe machining step, the lens blank is adhered or blocked onto a brass-chuck or poly(methylmethacrylate) cylinder. This may be achieved by using a low (melting) temperature blocking wax or water-ice where the blank is cryo-lathed below the freezing temperature of water. Depending on the cutting parameters employed, it may be desirable to cool the disc during lathing, to a temperature below its Tg in order to increase its hardness. Cooling may be provided by a cold-air stream, such as a vortex cold-air tube or cryogenic air-stream. Alternatively the disc may be cold-blocked, where the blocking chuck is cooled to an appropriate lathing temperature, typically below the freezing point of water, which also permits water-ice blocking. Additional benefit may also be gained through the use of a cryogenic lathing system where the actual cutting tool and the polymer are held at low temperatures during the cutting process.

After each ophthalmic lens surface has been lathe machined into the polymer, the machined surface may be inspected for defects and optical performance. Haptics may then be milled or fitted, depending on the ophthalmic lens design. Typically, the final ophthalmic lens is then inspected for defects.

For example, a typical method of lathe machining an IOL from a lens blank comprises one or more of the following steps:
(i) blocking a lens blank on a brass-chuck or a poly(methylmethacrylate) cylinder, for example using a low temperature blocking wax or ice-blocking using a cooled chuck and water as the adhesive agent;
(ii) applying a cold-airstream onto the rotating disc, such as by using a vortex cold-air tube, and lathe machine the first surface of a lens from a lens blank;
(iii) inspecting the machined surface for defects. If no defects are present, then de-block the machined lens blank;
(iv) blocking the first surface of the lens blank onto the chuck, for example using a low temperature blocking wax or ice-blocking using a cooled chuck and water as the adhesive agent;
(v) applying a cold-air stream onto the rotating disc, for example, by using a vortex cold-air tube. Then lathe the second surface of the lens optic from the lens blank;
(vi) inspecting the machined surface for defects;
(vii) milling the haptics e.g. for a one piece IOL design. A cold-air stream may be applied or alternatively the semi-finished IOL piece is held in position on a cryogenic plate with ice-blocking. For a multi-piece IOL design, the IOL haptics are attached;
(viii) de-blocking the IOL, for example by dissolving the blocking wax with 80-100 petroleum ether or allowing the water-ice to melt;
(ix) polishing the IOL to smooth the lens surfaces and the lens edges;
(x) hydrating the IOL in physiological saline, if required; and
(xi) inspecting the final IOL for defects.

Alternatively, step (x) may be performed prior to step (ix).

The invention also provides a method of preparing an ophthalmic lens of the invention, such as an IOL, by direct formation of a partial or complete lens using a mould designed specifically for that purpose. The method comprises the step of polymerising a polymerisable composition of the present invention in a mould in order to form an ophthalmic lens, wherein the mould is shaped so as to provide an ophthalmic lens having anterior and/or posterior portions consistent with conferring the desired optical performance (for example, focussing power) onto the polymer article.

As before, the polymerisable composition of the present invention may be polymerised in the mould to form the polymer. As before, an uncured polymer may be placed in the mould and cured, as an alternative method.

The mould design may encompass the anterior and/or posterior portion of the lens, or the complete lens. If only one lens surface is directly moulded, then the optics of the complementary surface may be subsequently formed by lathing and machine milling, either at room temperature or at a reduced temperature, as described above.

The mould design may encompass a single piece IOL design that incorporates moulded haptics or, alternatively, the haptics may be machined subsequent to the polymerisation and curing/or curing steps. Alternatively, a mould design may be used that is capable of providing a finished or semi-finished lens which is suitable for permanent attachment to haptic elements thereby to form a two or three-piece IOL design. Flushing and/or drying steps, as described above, may be included in the moulding of a partial or complete lens.

Where a partially finished lens is prepared from a moulding process, further machining steps are required to produce a complete lens. The precise machining steps to be carried out depend on what facets of the optic or haptics remain to be completed. For example, for a semi-finished lens shape with a completed first surface, a lathe-machining protocol such as the one described in steps (iv) to (xi) above may be followed. The steps generally described above for the lathe machining method can be used to machine a second complete surface and/or to mill or attach haptics to an ophthalmic lens that is moulded as a partially finished lens shape.

In another aspect of the inventions there is provided a method of forming a polymeric article by curing a linear polymer prepared from the polymerisable composition of the invention. The linear polymer is thus composed of polymeric units derived from the first monomer of the invention and optionally one or more polymeric units derived from the second, third and fourth monomers for use in the invention. The curing process may also be referred to as a crosslinking procedure.

In one embodiment, a polymer may be physically “cured” by the formation of an interpenetrating polymer network (IPN). Here the polymer is solubilised with a polymerisable monomer(s) which is/are polymerised to form a second polymer that is co-contingent with the first thereby to provide an interweaving polymer network which is essentially non-divisible (“intermingled”) due to chain entanglement. The polymers within the IPN formulation may optionally each incorporate cross-linking components so as to allow for the introduction of chemical cross-links.

In a further embodiment, a linear polymer may be formed comprising polymeric units derived from the first monomer of the invention. The linear polymer further comprises functionality that can be interlinked (“cured”) in a subsequent step. The functionality may be present in the polymeric units derived from the first monomer of the invention, and/or it may be present in one or more of the polymeric units derived from the second, third or fourth monomers, where present.

One example of a reactive monomer suitable for incorporation into a polymer that is to be cured is glycidyl methacrylate. When contained within a polymer, the epoxide functionality of this monomer is capable of forming interlinks with adjacent polymer chains by reaction with a suitable dinucleophile. Examples include, but are not limited to, an alkyldialkoxide, an alkyldimercaptan, an alkyldiamine, an alkyldicarboxylic acid, and an alkyldicarboxylate salt.

An alternative approach is to incorporate into a linear polymer functionality that is capable of photo-crosslinking. One example of a photoreactive monomer suitable for incorporation into a polymer that is to be cured is 9-anthracene methyl methacrylate. When contained within a polymer, the photoreactive moiety undergoes light-induced 4π+4π cycloadditon with an adjacent anthracene ring to form a dianthracene linkage.

Where a polymer contains aryl groups, the aryl groups may be crosslinked by reaction with a dihalogen compound under electrophilic aromatic substitution conditions, for example using the Friedel-Craft alkylation acylation reaction.

Aryl groups in a polymer may be reacted under Blanc conditions to generate the appropriate arylmethylene chlorides. The chloromethyl groups may be reacted with a dinucleophile to form the crosslinks. Suitable dinucleophiles include, but are not limited to, alkyldialkoxide, alkyldimercaptan, alkyldiamine, alkyldicarboxylic acid, and alkyldicarboxylate salt. Alternatively the chloromethyl groups may be reacted with the potassium salt of maleimide and the resultant arylmethylenemaleimide may be permitted to undergo cross-linking via a photo-crosslinking mechanism and/or free-radical vinyl-type polymerisation, as appropriate.

### Absorption Properties

Typically each of the two aryl-rings of the monomer (I) absorbs a negligible amount of radiation within the wavelength range 300-900 nm. The aryl ring substituents, where present, are selected such that a polymer, blank or lens comprising these moieties absorbs a negligible amount of light at wavelengths in the 300-900 nm range.

Any other aryl groups present in the monomer molecule, or present in the polymerisable composition, may also absorb light at a negligible amount at a wavelength in the range 300-900 nm. However, where the polymerisable composition comprises UV-blocker components, such components are provided specifically to absorb and dissipate radiative energy within the UVA and UVB spectral ranges.

Preferably the monomer (I) itself absorbs a negligible amount of light having a wavelength in the range 300-900 nm. Similarly, a polymer prepared from a polymerisable composition comprising the monomer absorbs a negligible amount of light having a wavelength in the range 300-900 nm (with the aforementioned exception of any UV-blocker monomer component). Thus, the monomer (also referred to as the first monomer) and the second, third and fourth monomers, where present, and other polymerization additives, where present, and the diluent, where present, absorb a negligible amount of light having a wavelength in the range 300-900 nm.

In one embodiment, each or both of the optionally substituted aryl rings of formula (I), or the monomer (I), or the polymer, do not significantly absorb light at a wavelength in the range 300 to 900 nm.

In one embodiment, the wavelength is selected from the range 300 to 400 nm.

In one embodiment, the wavelength is selected from the range 320 to 400 nm.

In one embodiment, the wavelength is selected from one or more of 310, 320, 330, 340, 350, 400, 450, 500, 550, 600, 700, 800 or 900 nm.

In one embodiment, each or both of the optionally substituted phenyl rings of formula (I), or the monomer (I), or the polymer, has a transmittance of at least 60%, at least 70%, at least, 80%, or at least 90% at the wavelength specified.

The phrase significantly absorb light may be taken to refer to the wavelength at which the group, monomer or polymer in question has its maximum UV absorbance (or minimum UV transmittance). In some embodiments, therefore, where the maximum UV absorbance lies outside the range 300 to 900 nm (for example, 200 to 300 nm), that group, monomer or polymer may be considered not to significantly absorb light at a wavelength in the range 300 to 900 nm.

### EMBODIMENTS

Various further embodiments of the invention are set out below. Each and every compatible combination of the embodiments described is explicitly disclosed herein, as if each and every combination was individually and explicitly recited.

The embodiments described below apply to the monomer compound of formula (I) and the polymer compound comprising units of formula (II), where appropriate.

In one embodiment, —R¹ is independently —H or alkyl. The alkyl may be C₁₋₆ alkyl.

In one embodiment, —R¹ is independently —H or -Me. Preferably, —R¹ is independently —H.

Where —R¹ is —H, the monomer or polymer may be referred to as an acrylate-based monomer or polymer. Where —R¹ is -Me, the monomer or polymer may be referred to as an methacrylate-based monomer or polymer.

In one embodiment, —Z— is independently —O—.

In one embodiment, —Z— is independently —NH— or —N(R)—.

The group —R is optionally substituted alkyl or aryl. The alkyl group may be C₁₋₆ alkyl. The aryl group may be C₅₋₁₀ aryl.

In one embodiment, —R is independently optionally substituted alkyl.

In one embodiment, —R is independently alkyl.

In one embodiment, —R is independently -Me or -Et.

In one embodiment, each of -Ar¹ and -Ar² is independently optionally substituted C₅₋₁₀ aryl.

In one embodiment, each of -Ar¹ and -Ar² is independently optionally substituted phenyl.

In one embodiment, each of -Ar¹ and -Ar² is optionally substituted with one or more substituents selected from the group consisting of C₁₋₆ alkyl, C₁₋₆ alkoxy, —C₁₋₆ haloalkyl, halo and —N(R′)₂, where each R¹ is H or C₁₋₆ alkyl.

In one embodiment, R¹ is C₁₋₆ alkyl.

In one embodiment, halo is selected from the group consisting of —F, —Cl and —Br.

In one embodiment, —C₁₋₆ haloalkyl is —CF₃.

In one embodiment, each of -Ar¹ and -Ar² is optionally substituted with one or more substituents, such as 1 to 5 substituents, selected from C₁₋₆ alkyl and C₁₋₆ alkoxy.

In one embodiment, each of -Ar¹ and -Ar² is optionally substituted with C₁₋₆ alkyl.

In one embodiment, each of -Ar¹ and -Ar² is optionally substituted with C₁₋₆ alkoxy.

In one embodiment, each of -Ar¹ and -Ar² is optionally substituted with 1, 3 or 5 substituents; 1, 2 or 3 substituents; 1 or 2 substituents; or 1 substituent.

In one embodiment, each of -Ar¹ and -Ar² is phenyl optionally mono-substituted at the 4-position.

The group —R² is —H, alkyl or aryl. The alkyl group may be C₁₋₆ alkyl. The aryl group may be C₅₋₁₀ aryl.

In one embodiment, —R² is independently —H or alkyl.

The group —R² may be alkyl or aryl where the corresponding monomer with —R⁴ is —H is susceptible to oxidation.

In one embodiment, —R² is independently —H or -Me. Preferably, —R⁴ is independently —H.

In one embodiment, each optionally substituted group is unsubstituted.

In one embodiment, each optionally substituted group is optionally substituted with one or more groups selected from halo, alkyl, aryl, heterocyclyl, arylalkyl, heterocycyl-alkyl, alkoxy, aryloxy, and alkylaryl.

In one embodiment, each optionally substituted group is optionally substituted with one or more groups selected from halo, alkyl, heterocyclyl, arylalkyl, heterocycyl-alkyl, and alkoxy.

In one embodiment, the polymerizable composition comprises the monomer of formula (I) (the first monomer) in an amount from 20 to 75 wt %, such as from 25 to 65 wt %, and a second monomer in an amount from 15 to 75 wt % such as from 15 to 65 wt %, optionally together with third and fourth monomers and other additives, as described herein.

In one embodiment, the polymerizable composition optionally comprises second, third and fourth monomers, and each monomer has (alkyl)acrylate functionality for polymerization with the first monomer.

### DEFINITIONS

Substituents are defined and exemplified below.

The phrase “optionally substituted” as used herein, pertains to a parent group which may be unsubstituted or which may be substituted.

Unless otherwise specified, the term “substituted” as used herein, pertains to a parent group which bears one or more substituents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known. The substituents may be selected from the groups listed below.

Alkyl: The term “alkyl” as used herein, pertains to a monovalent moiety obtained by removing a hydrogen atom from a carbon atom of a saturated hydrocarbon compound, which may be aliphatic or alicyclic (cycloalkyl). The alkyl group may be a C₁₋₂₀, C₁₋₁₂, C₁₋₆, C₃₋₆, C₁₋₂₀, C₃₋₁₂, C₃₋₅, C₃₋₅, C₁₋₄ or C₁₋₂ alkyl group. A preferred aliphatic alkyl group is C₁₋₆ alkyl, most preferably C₁₋₄ alkyl. A preferred cycloalkyl group is C₃₋₅ cycloalkyl, most preferably C₅₋₆ cycloalkyl.

An aliphatic alkyl group may be linear or branched.

Examples of alkyl groups include, but are not limited to, methyl (C₁), ethyl (C₂), propyl (C₃), butyl (C₄), pentyl (C₅), hexyl (C₆).

An example of a substituted alkyl group includes, but is not limited to, perfluorooctyl (C₆F₁₃). Such a group may be referred to as a haloalkyl group, such as described herein.

Examples of linear alkyl groups include, but are not limited to, methyl (C₁), ethyl (C₂), n-propyl (C₃), n-butyl (C₄), n-pentyl (amyl) (C₅), n-hexyl (C₆).

Examples of branched alkyl groups include iso-propyl (C₃), iso-butyl (C₄), sec-butyl (C₄), tert-butyl (C₄), iso-pentyl (C₅), and neo-pentyl (C₆).

Examples of cycloalkyl groups include, but are not limited to, those derived from: saturated monocyclic hydrocarbon compounds:

cyclopropane (C₃), cyclobutane (C₄), cyclopentane (C₆), cyclohexane (C₆), methylcyclopropane (C₄), dimethylcyclopropane (C₅), methylcyclobutane (C₅), dimethylcyclobutane (C₆), and methylcyclopentane (C₆).

Alkenyl: The term “alkenyl” as used herein, pertains to a monovalent moiety obtained by removing a hydrogen atom from a carbon atom of an unsaturated hydrocarbon compound having one or more carbon-carbon double bonds, which may be aliphatic or alicyclic (cycloalkenyl). The alkenyl group may be a C₂₋₆ or C₃₋₆ alkenyl group.

Examples of alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

An example of a substituted alkenyl group includes, but is not limited to, styrene (—CH═CHPh or —C(Ph)=CH₂).

Examples of cycloalkenyl groups include, but are not limited to, those derived from cyclopropene (C₃), cyclobutene (C₄), cyclopentene (C₅), cyclohexene (C₆), methylcyclopropene (C₄), dimethylcyclopropene (C₅), methylcyclobutene (C₆), dimethylcyclobutene (C₆), and methylcyclopentene (C₆).

Alkynyl: The term “alkynyl” as used herein, pertains to a monovalent moiety obtained by removing a hydrogen atom from a carbon atom of an unsaturated hydrocarbon compound having one or more carbon-carbon triple bonds, which may be aliphatic or alicyclic (cycloalkynyl). The alkynyl group may be a C₂₋₆ or C₃₋₆ alkynyl group.

Examples of alkynyl groups include, but are not limited to, ethynyl (—C≡CH) and 2-propynyl (propargyl, —CH₂—C≡CH).

Amino: —NR¹R², wherein R¹ and R² are independently amino substituents, for example, hydrogen, an alkyl group (also referred to as alkylamino or dialkylamino), an alkenyl group, an alkynyl group, a heterocyclyl group, or an aryl group, preferably H or an alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino. The amino group may be —N(R′)₂, where each R′ is H or C₁₋₆ alkyl.

Arylalkyl: The term “arylalkyl” or “aralkyl”, as used herein, pertains to a monovalent moiety obtained by removing a hydrogen atom from a carbon atom of an alkyl group that is covalently bonded to an aromatic ring. The alkyl and aryl part of the group are as defined above. The arylalkyl group may be C₆₋₂₁, C₆₋₁₃, C₆₋₈, or C₆₋₇ arylalkyl group.

The prefixes (e.g. C₆₋₂₁, C₆₋₁₃, C₆₋₈, etc.) denote the number of carbon atoms in the alkyl group and the total number of ring atoms. For example, the term “C₈ arylalkyl” as used herein, pertains to an arylalkyl group where the aryl group has 5 or 6 ring atoms and the alkyl chain has 2 or 3 carbon atoms. Typically the alkyl group has 1 or 2 carbon atoms. An example of an arylalkyl group includes, but is not limited to, benzyl (—CH₂Ph).

Alkylaryl: The term “alkylaryl” as used herein, pertains to a monovalent moiety obtained by removing a hydrogen atom from a carbon atom of an aryl group that is covalently bonded to an alkyl group. The alkyl and aryl part of the group are as defined above. The alkylaryl group may be C₆₋₂₁, C₆₋₁₃, C₆₋₈, or C₆₋₇ arylalkyl group.

The prefixes (e.g. C₆₋₂₁, C₆₋₁₃, C₆₋₈, etc.) denote the number of carbon atoms in the alkyl group and the total number of ring atoms. For example, the term “C₈ alkylaryl” as used herein, pertains to an alkylaryl group where the aryl group has 5 or 6 ring atoms and the alkyl chain has 2 or 3 carbon atoms. Typically the alkyl group has 1 or 2 carbon atoms. An example of an arylalkyl group includes, but is not limited tolyl (-PhMe).

Aryl: The term “aryl”, as used herein, pertains to a monovalent moiety obtained by removing a hydrogen atom from an aromatic ring atom of an aromatic compound. It is preferred that aryl groups present in the monomers, compositions and polymers of the invention absorb a negligible amount of light having a wavelength in the range 300-900 nm. The aryl group may be a C₅₀₁₂, C₅₋₁₀ or C₅₋₆ aryl group.

In this context, the prefixes denote the number of ring atoms, or range of number of ring atoms, whether carbon atoms or heteroatoms. For example, the term “C₅₋₆ aryl” as used herein, pertains to an aryl group having 5 or 6 ring atoms.

The term aryl may refer to a carboaryl or heteroaryl group. The ring atoms may be all carbon atoms, as in a carboaryl group, such as C₆₋₁₀ carboaryl. Examples of carboaryl groups include, but are not limited to, phenyl (C₆) and napthyl.

Alternatively, the ring atoms may include one or more heteroatoms, as in a heteroaryl group, such as C₅₋₁₂, C₅₋₁₀ or C₅₋₆ heteroaryl. Examples of monocyclic C₅₋₆ heteroaryl groups include, but are not limited to, those derived from:
N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Ether: —OR, wherein R is an ether substituent, for example, an alkyl group (referred to as alkoxy), an arylalkyl group, an alkenyl group, an alkynyl group, a heterocyclyl group, or an aryl group (referred to as aryloxy), preferably an alkyl group, an arylalkyl group, or an aryl group. Examples of ether groups include, but are not limited to, —OCH₃, —OCH₂CH₃, —O-t-Bu, —OBn, and —OPh.

Halo: —F, —Cl, —Br, and —I.

Haloalkyl group: The term “haloalkyl,” as used herein, pertains to an alkyl group, such as an alkyl group described herein, in which at least one hydrogen atom (e.g., 1, 2, 3) has been replaced with a halogen atom (e.g., F, Cl, Br, I). If more than one hydrogen atom has been replaced with a halogen atom, the halogen atoms may independently be the same or different. Every hydrogen atom may be replaced with a halogen atom, in which case the group may conveniently be referred to as a C perhaloalkyl group. Examples of such groups include, but are not limited to, —CF₃, —CHF₂, —CH₂F, —CCl₃, —CBr₃, —CH₂CH₂F, —CH₂CHF₂, and —CH₂CF₃.

Heterocyclyl: The term “heterocyclyl” as used herein, pertains to a monovalent moiety obtained by removing a hydrogen atom from a ring atom of a heterocyclic compound. The heterocyclyl group may be a C₃₋₂₀ heterocyclyl group of which from 1 to 10 are ring heteroatoms, a C₃₋₇ heterocyclyl group of which from 1 to 4 are ring heteroatoms, or a C₅₋₆ heterocyclyl group of which 1 or 2 are ring heteroatoms. In one embodiment, the heterocyclyl group is a C₃ heterocyclyl group. In one embodiment, the heterocyclyl group is epoxy. In one embodiment, the heterocyclyl group is obtained by removing a hydrogen atom from a ring carbon atom of a heterocyclic compound.

In one embodiment, the heteroatoms may be selected from O, N or S. In one embodiment the heterocyclyl group is obtained by removing a hydrogen atom from a ring nitrogen atom, where present, of a heterocyclic compound. The heterocyclyl group may be a C₃₋₂₀, C₃₋₇, or C₅₋₆ heterocyclyl group.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆ heterocyclyl”, as used herein, pertains to a heterocyclyl group having 5 or 6 ring atoms.

Examples of monocyclic heterocyclyl groups include, but are not limited to, those derived from:

N₁: piperidine (C₆);
O₁: pyran (C₆);
N₂: piperazine (C₆);
O₂: dioxane (C₆);
N₁O₁: morpholine (C₆);

Heterocyclyl-alkyl: The term “heterocyclyl-alkyl”, as used herein, pertains to a monovalent moiety obtained by removing a hydrogen atom from a carbon atom of alkyl group that is covalently bonded to a heterocyclic compound. The heterocyclic ring or heterocyclyl group is as defined above and may have from 3 to 20 ring atoms, of which from 1 to 10 are ring heteroatoms. Preferably, each ring has from 3 to 7 ring atoms, of which from 1 to 4 are ring heteroatoms.

In this context, the prefixes (e.g. C₆₋₇ etc.) denote the number of carbon atoms in the alkyl group and the total number of ring atoms, whether carbon atoms or heteroatoms. For example, the term “C₆₋₇ heterocyclyl”, as used herein, pertains to a heterocyclyl group having 5 or 6 ring atoms and an alkyl group having 1 or 2 carbon atoms.

### Other Preferences

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### EXAMPLES

### Example 1

### Synthesis of Di(Benzyl)Methanol (DBM)

Absolute ethanol (300 mL) was added to a 1 L 2-neck RB flask and a B19 double-layer coil condenser was attached to the side-arm and this condenser was in turn connected to a vacuum-nitrogen manifold and the apparatus purged with a fast flow of nitrogen. 4×2.0 g and 1×1.0 g aliquots of sodium borohydride were added at intervals to the ethanol forming a slightly turbid colourless solution. Separately 1,3-diphenylacetone (50.0 g, 238 mmol) was dissolved in 100 mL of warm ethanol and this was poured into a 250 mL pressure-equalising addition funnel attached to the top neck of the reaction flask. The flask that had contained the 1,3-diphenylacetone/ethanol solution was washed out with a 25 mL portion of ethanol into the pressure-equalising addition funnel. The 1,3-diphenylacetone solution was then added dropwise to the sodium borohydride/ethanol solution at room temperature over a period of 60 minutes, during which a mild exotherm was observed. At the completion of the addition the pressure-equalising addition funnel was then washed out with 25 mL of absolute ethanol into the reaction mixture which was then stirred for 18 hours at room temperature under a nitrogen atmosphere to complete the reaction.

The reaction mixture was quenched with 100 mL de-ionised water which deposited a white “lump”. The solution was transferred to a 1 L Florentine flask and the white “lump” that remained in the reaction flask was dissolved in 100 mL of warm water (heat-gun) forming a slightly turbid soln. The reaction solvent in the Florentine flask was then driven off in vacuo (rotary evaporator) yielding a colourless liquid containing a small amount of white semi-solid. The florentine flask was then removed from the rotary evaporator and the mixture allowed to cool to RT before the aqueous solution formed from the dissolution of the white “lump” in the quenched reaction mixture was poured into the flask together with 250 mL diethyl ether and the entire mixture transferred to a 500 mL separating funnel and the mixture shaken for 2-minutes. The lower aqueous layer was then partitioned and separated and the upper ethereal layer extracted with 2×200 mL portions of brine before being partitioned and collected in a 500 mL Erlenmeyer flask. The diethyl ether was then dried over anhydrous sodium sulfate for a period of 30 minutes. The drying solution was then filtered and the collected solid washed with 2×30 mL portions of diethyl ether. The filtrate and washings were combined (colourless solution) and evaporated to dryness in vacuo (rotary evaporator) yielding a slightly viscous colourless liquid which was then fractionally distilled in vacuo through a Claisen head:

FRACTION #1: 101° C.-114° C. (0.128 torr-0.119 torr)—Colourless liquid (discarded)
FRACTION #2: 114° C.-120° C. (0.119 torr-0.105 torr)—Colourless liquid

Fraction #1 consisting of only a few milliliters of material was discarded whereas fraction #2 was retained for characterisation and a yield taken. Yield: 45.926 g (90.98%); colourless liquid).

### Example 2

### Synthesis of Di(Benzyl)Methyl Acrylate (DBMA)

Di(benzyl)methanol (44.0 g, 207.3 mmol) was weighed into a 500 mL 3-neck RB flask to which was connected a suba-seal (side-arm), a 125 mL pressure-equalising addition funnel (side-arm) stoppered with a suba-seal, and a cone-tubing adaptor (centre-socket) which was connected in turn to a nitrogen-vacuum manifold and the apparatus purge-filled with nitrogen twice. Dichloromethane (200 mL, anhydrous) was cannula-transferred into the reaction flask forming a colourless solution. Hunig's base (48.5 mL, 278.4 mmol) was added to the reaction mixture via a 20 mL disposable gastight syringe (2×20 mL and 1×8.5 mL portions). Deinhibited [by distillation under nitrogen] acryloyl chloride (22.25 mL; 273.8 mmol) was then added to the pressure-equalising addition funnel via a Hamilton gastight syringe followed by anhydrous dichloromethane (75.0 mL). The reaction flask was then surrounded with a dry-ice/acetone cooling bath and the reaction mixture allowed to cool to −78° C. prior to the dropwise addition of the acryloyl chloride/dichloromethane solution over a period of 60 minutes under an inert nitrogen atmosphere during which time a dense white precipitate ([DIPEA]HCl) deposited from the reaction mixture. The reaction mixture was then stirred in the cooling bath and allowed to warm slowly to room temperature under nitrogen over a period of 18 hours.

The reaction flask containing a deep orange solution was then surrounded with a water-ice/water cooling bath and the reaction mixture cooled to <+5° C. before 50 mL methanol was added dropwise to quenched the excess acryloyl chloride, this was accompanied by a slight exotherm (reaction mixture temperature increased to +5° C. before falling back). The quenched reaction mixture was then transferred to a 1 L separating funnel and extracted with; (i) 330 mL 1M HCl (aq.); (ii) 400 mL aqueous saturated sodium hydrogen carbonate solution; (iii) 400 mL brine. The extraction mixture was then partitioned and separated and the lower organic layer collected and dried over anhydrous magnesium sulfate for a period of 45 minutes. The drying mixture was then filtered and the collected drying agent washed with 2×25 mL portions of dichloromethane. The filtrate and washings were combined and stripped to dryness in vacuo (rotary evaporator) to yield a slightly viscous orange liquid which was characterised by GC-MS; this indicated that complete conversion to DBMA monomer had occurred forming the monomer in 99.5% purity (by integration of the gas chromatogram). The crude product was then fractionally distilled in vamp in a 50 mL Claisen flask with a 5 cm Vigreux column over one Chattaway spatula measure of N,N′-dinapthyl-p-phenylenediamine (DNPD) and two Chattaway spatula measures of N,N′-diphenyl-p-phenylenediamine (DPPD):

FRACTION #1: 40° C.-42° C. (0, 0 torr-0.183 torr)—colourless liquid
FRACTION #2: 42° C.-119.5° C. (0.183-0.165 torr)—pale yellow liquid
FRACTION #3: 119.5° C.-123° C. (0.165-0.141 torr)—pale yellow liquid

Fractions #1 & #2 were discarded. Fraction #3, the main fraction, was taken up in pentane (150 mL; mixed isomers) and extracted with 2×200 mL 2 M HCl (aq) and 2×200 mL water to remove any co-distilled DPPD inhibitor. Post extraction the layers were partitioned and separated and the pentane/DBMA solution isolated and dried over anhydrous sodium sulfate for a period of 60 minutes before the drying mixture was filtered and the collected solids washed with 2×30 mL portions of pentane (mixed isomers). The filtrate and washings were combined in a 500 mL Florentine flask and 4-methoxyphenol (0.0025 g) was added in order to inhibit the DBMA monomer to a level of 50 ppm (MEHQ) prior to the pentane solvent being stripped off in vacuo (rotary evaporator). The monomer was then further dried using a vacuum manifold (pressure: <0.20 torr; flask wrapped with Al-foil) at room temperature for 20 hours whilst magnetically stirring the DBMA monomer with a small stirrer follower. Next day the DBMA monomer was a free flowing very slightly “off-white” liquid. The monomer was syringe filtered (glass microfibre) into a 60 mL storage bottle and a yield taken. Yield: 48.2 g [87.3%; “off-white” transparent liquid].

¹H NMR (300 MHz, CDCl₃): δ 2.8-3.0 ppm (m, 4H, —CH₂— [×2]), 5.38 (p, 1H, —O—C(H)<), 5.76 (dd, 1H, acrylate-H), 6.05 (dd, 1H, acrylate-H), 6.32 (dd, 1H, acrylate-H), 7.15-7.33 (m, 10H, -PhH [×2]).

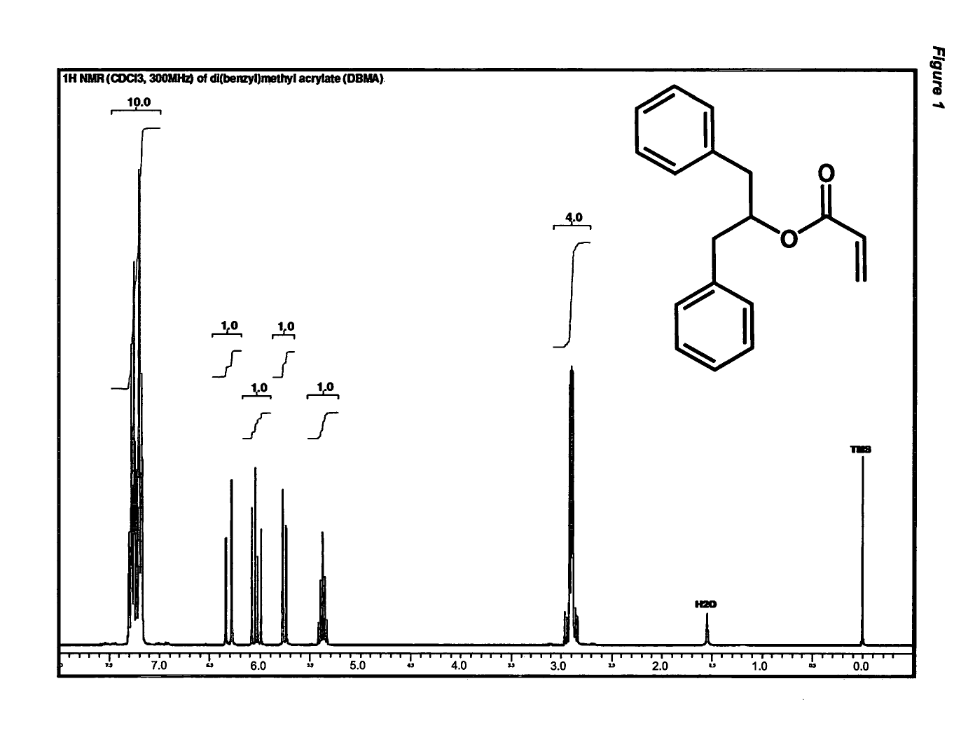

FIG. 1 shows the ¹H NMR (CDCl₃, 300 MHz) spectrum of the distilled di(benzyl)methyl acrylate (DBMA) monomer.

### Examples 3-9

### Monomer Formulations and Photo-Polymerisation

The formulations presented in Table 1 were prepared in the following manner. The monomers and photoinitiator were removed from their respective storage freezer, fridge, cabinet and equilibrated to ambient temperature over a period of 60 minutes. The monomer components were then mixed in the ratios specified in Table 1, thoroughly mixed by magnetic stirring at a rate of 700 rpm for a period of 5 minutes before the vessel containing the formulations was wrapped with aluminium foil and the photoinitiator added in the proportion designated in Table 1. The vessel containing the formulation was then covered so as to completely exclude all light and the mixture stirred vigorously at 700 rpm for a period of 30 minutes until complete dissolution of all the components had occurred. The formulation was then injected through a 0.45 μm syringe filter and the filtrate used to fill standard polypropylene 1.0 mm (depth)×12.0 mm (diameter) cylindrical disc moulds, with individual attachable polypropylene caps, which had been degassed by vacuum purging at <1 torr for 60 minutes and then stored under a nitrogen atmosphere. The filled moulds were then placed in a BINDER APT.line™ FP115 forced convection oven thermally equilibrated to 65° C. and photopolymerised 15 mm directly underneath a rectangular array of 4× Philips TL-D K 30W Actinic BL fluorescent tubes for a period of 30 minutes. At the completion of the polymerisation period the moulds were removed from the BINDER FP115 oven and allowed to cool to room temperature for a period of 15 minutes before being cold-extruded by placing in a 500 mL beaker half-filled with dry-ice and cooling for a period of one minute prior to removing the mould-cap with forceps, inverting the mould and evenly tapping the underside of the mould with a small plastic mallet until the polymer disc cleanly detached from the mould.

The mechanical parameters recorded in Table 1 were measured on dry samples of the polymer as were the tack determinations and assessments of folding/unfolding properties. The haze and refractive index measurements were made on sample of polymer that had been immersed in de-ionised water at 45° C. for a period of at least 24 hours before being allowed to equilibrate to room temperature for a period of 2 hours prior to characterisation.

The results show that is advantageous to use a combination of aryl-containing monomers (e.g. DBMA, DPEMA and PEA) as such can produce polymer products with the most desirable characteristics. Thus, monomers such as HEA and HBA are less preferred. HEA, for example, does not introduce a sufficiently low Tg for the overall polymer. HBA may be used to alter the Tg value to an appropriate level, but is use is associated with slight hazing in the polymer product.

TABLE 1
Composition and Properties of Example Polymers 3-9

| | Example Number | | | | | | |
|---|---|---|---|---|---|---|---|
| | 3 | 4 | 5 | 6 | 7 | 8 | 9 |
| Formulation (wt %) | | | | | | | |
| DBMA | 39.0 | 39.0 | 25.0 | 35.0 | 42.5 | 50.0 | 60.0 |
| DPEMA | | | 60.0 | | | | |
| PEA | 39.0 | 39.0 | | 50.0 | 42.5 | 35.0 | 25.0 |
| HEA | 20.0 | | | | | | |
| HBA | | 20.0 | | | | | |
| PEG(700)DA | | | 15.0 | 15.0 | 15.0 | 15.0 | 15.0 |
| BDDA | 2.0 | 2.0 | | | | | |
| IRG-819 | 0.20 | 0.20 | 0.20 | 0.20 | 0.20 | 0.20 | 0.20 |

Optical Properties

| | | | | | | | |
|---|---|---|---|---|---|---|---|
| Polymer Colour | Colour- | Colour- | Colour- | Colour- | Colour- | Colour- | Colour- |
| | less | less | less | less | less | less | less |
| Hydrated | Visible | Very | Visible | Haze- | Haze- | Haze- | Haze- |
| Polymer Haze | | Hazy | | free | free | free | free |
| n_{D}²⁰ (Hydrated) | 1.5535 | 1.5523 | 1.5605 | 1.5520 | 1.5547 | 1.5567 | 1.5555 |

Mechanical Properties (Dry Polymer)

| | | | | | | | |
|---|---|---|---|---|---|---|---|
| Dry Polymer Tack | Minimal | Slight | Slight | Medium | Medium | Low | Very low |
| | | | | | to Low | | |
| “In Hand” | Poor | Firm but | Easily | Easily | Easily | Foldable | Firm but |
| Foldability | | pliable | foldable | foldable | foldable | | foldable |
| Unfolding Time | >60 s | >30 s | <10 s | <10 s | <10 s | ≦20 s | ≦25 s |
| (s at 23° C.) | | | | | | | |
| E-Modulus (MPa) | 1.90 | 3.40 | 3.37 | 1.97 | 2.51 | 3.51 | 6.51 |
| Tensile Strength | 3.07 | 3.72 | 3.63 | 2.47 | 4.52 | 5.00 | 4.07 |
| (MPa) | | | | | | | |
| Elongation (mean) | 457 | 269 | 194 | 159 | 230 | 247 | 207* |
| (%) | | | | | | | |
| Elongation (max) | 497 | 292 | 209 | 200 | 249 | 271 | 234* |
| (%) | | | | | | | |

*The polymer strip did not break but instead pulled out of tensiometer grips.

### Monomers:

DBMA di(benzyl)methyl acrylate
DPEMA di(phenylethyl)methyl acrylate
PEA 2-phenylethyl acrylate
HEA 2-hydroxyethyl acrylate
HBA 4-hydroxybutyl acrylate
PEG(700)DA poly(ethyleneglycol) diacrylate [M_{w} PEG chain: 700]
BDDA 1,4-butanediol diacrylate

### Photoinitiator:

IRG-819 IRGACURE-819 [bis(2,4,6-trimethylbenzoyl)phenylphosphine oxide]

### Examples 10-14

### Solvent Extraction of Polymeric Materials

The 12.00 mm (diameter)×1.00 mm (depth) cylindrical polymeric discs formed from the monomeric formulations presented in Table 2 [examples 5-9 of Table 1] were subjected to exhaustive solvent extraction through a continuous Soxhlet process using an acetone/n-hexane mixture, refluxed for a period of 24 hours under an inert nitrogen atmosphere, whilst containing the discs within white lens baskets to stop the polymer samples adhering to each other. After the completion of the 24 hour extraction period the Soxhlet apparatus was allowed to cool to room temperature before the lens baskets containing the extracted discs were removed and allowed to dry in air in a fume-hood for a period of at least 7 hours before being transferred to a vacuum oven and subjected to the following conditions [(i) vacuum for 24 hr at 25° C.; (ii) ramp to 60° C. at 7° C./hr; (iii) hold at 60° C. for 72 hr; (iv) ramp down to 30° C. at 10° C./hr] with an ultimate vacuum pressure ≦0.1 torr obtained. The polymer discs were weighed on a 4-decimal place balance prior to extraction and after the extraction/drying process to permit a determination of the gravimetric residuals within the polymer matrix post photo-polymerisation; these are presented in Table 2. The post-extraction/drying mechanical parameters were also determined using a Zwick Z0.5 tensiometer.

TABLE 2
Composition and Properties of Example Polymers 10-14

| | Example Number | | | | |
|---|---|---|---|---|---|
| | 10 | 11 | 12 | 13 | 14 |
| Formulation (wt %) | | | | | |
| DBMA | 25.0 | 35.0 | 42.5 | 50.0 | 60.0 |
| DPEMA | 60.0 | | | | |
| PEA | | 50.0 | 42.5 | 35.0 | 25.0 |
| PEG(700)DA | 15.0 | 15.0 | 15.0 | 15.0 | 15.0 |
| IRG-819 | 0.20 | 0.20 | 0.20 | 0.20 | 0.20 |

Solvent Extraction Processing

| | | | | | |
|---|---|---|---|---|---|
| Extraction Method | Soxhlet | Soxhlet | Soxhlet | Soxhlet | Soxhlet |
| Solvents | DMK/hex | DMK/hex | DMK/hex | DMK/hex | DMK/hex |
| Solvent Ratio | 59:41 | 59:41 | 59:41 | 59:41 | 59:41 |
| Extraction Period (h) | 24 | 24 | 24 | 24 | 24 |
| Post Extraction Drying | Vacuum | Vacuum | Vacuum | Vacuum | Vacuum |
| Method | oven | oven | oven | oven | oven |
| Drying Temperature | 60 (72 hr) | 60 (72 hr) | 60 (72 hr) | 60 (72 hr) | 60 (72 hr) |
| (° C.) | | | | | |
| Gravimetric Residuals | 4,889 | 3,870 | 3,398 | 3,214 | 3,564 |
| (ppm) | | | | | |

Mechanical Properties (Extracted Dry Polymer)

| | | | | | |
|---|---|---|---|---|---|
| E-Modulus (MPa) | 4.87 | 2.32 | 2.94 | 3.81 | 5.50 |
| Tensile Strength (MPa) | 5.34 | 3.23 | 4.64 | 4.72 | 3.72 |
| Elongation (mean) (%) | 217 | 197 | 223 | 226 | 196* |
| Elongation (max) (%) | 247 | 223 | 257 | 267 | 212* |

*Polymer strip did not break but instead pulled out of tensiometer grips.

### Monomers:

DBMA di(benzyl)methyl acrylate
DPEMA di(phenylethyl)methyl acrylate
PEA 2-phenylethyl acrylate
HEA 2-hydroxyethyl acrylate
HBA 4-hydroxybutyl acrylate
PEG(700)DA polyethyleneglycol) diacrylate [M_{w} PEG chain: 700]
BDDA 1,4-butanediol diacrylate

### Photoinitiator

IRG-819 IRGACURE-819 [bis(2,4,6-trimethylbenzoyl)phenylphosphine oxide]

### Additional Examples

Further example polymerizable compositions and polymers were prepared according to the methods described above. The polymers of examples 15-19 were prepared in the same manner as the polymers 3-14 in the examples above.

The polymers prepared from polymerizable compositions 15-18, shown in Table 3 below, are suitable for comparison with the polymers of examples 3-8, set out in Table 1 above.

Examples 5-9 above show that an increased content of DBMA is associated with a reduction in haze levels, and an increased content of BPPA is also associated with an advantageous increase in other characterises such as tack, modulus, elongation, and unfolding characteristics (see Table 1). Where a polymer does not include DBMA, and has a high content of DPEMA, the modulus and elongation values are reduced compared with those compositions containing DBMA and DPEMA (compare Examples 15 and 16 in Table 3 with Example 17 in Table 3 and Example 5 in Table 1).

Examples 5 and 17 in Tables 1 and 3 are seen to have a visible haze. However, it can be seen that increased DBMA quantities are associated with a reduction in the haze levels, thus the presence of DBMA is advantageous.

The use of PEA in place of DBMA is associated with a reduced E-modulus and reduced tensile strength. See Example 18 compared with Examples 5 to 8 in Table 1,

TABLE 3
Composition and Properties of Example
and Reference Polymers 15-18

| | Example Number | | | |
|---|---|---|---|---|
| | 15 | 16 | 17 | 18 |
| Formulation (wt %) | | | | |
| DBMA | | | 30.0 | |
| DPEMA | 85.0 | 87.50 | 55.0 | 75.0 |
| PEA | | | | 15.0 |
| HEA | | | | |
| HBA | | | | |
| PEG(700)DA | 15.0 | 12.50 | 15.0 | 10.0 |
| BDDA | | | | |
| SRG-819 | 0.20 | 0.20 | 0.20 | 0.20 |

Optical Properties

| | | | | |
|---|---|---|---|---|
| Polymer Colour | Colour- | Colour- | Colour- | Colour- |
| | less | less | less | less |
| Hydrated | Minimal | Minimal | Visible | Visible |
| Polymer Haze | | | | |
| n_{D}²⁰ (Hydrated) | 1.5598 | 1.5610 | 1.5629 | 1.5620 |

Mechanical Properties (Dry Polymer)

| | | | | |
|---|---|---|---|---|
| Dry Polymer Tack | Colour- | Colour- | Colour- | Colour- |
| | less | less | less | less |
| “In Hand” | Minimal | Minimal | Visible | Visible |
| Foldability | | | | |
| Unfolding Time | 1.5598 | 1.5610 | 1.5629 | 1.5620 |
| (s at 23° C.) | | | | |
| E-Modulus (MPa) | Colour- | Colour- | Colour- | Colour- |
| | less | less | less | less |
| Tensile Strength | Minimal | Minimal | Visible | Visible |
| (MPa) | | | | |
| Elongation (mean) | 1.5598 | 1.5610 | 1.5629 | 1.5620 |
| (%) | | | | |
| Elongation (max) | Colour- | Colour- | Colour- | Colour- |
| (%) | less | less | less | less |

Example 19 was prepared in the same manner as Examples 10-14, and the composition of the polymer and its properties are set out in Table 4 below.

TABLE 4
Composition and Properties of Example Polymers 19

| | Example Number |
|---|---|
| | 19 |
| Formulation (wt %) | |
| DBMA | |
| DPEMA | 85.0 |
| PEA | |
| PEG(700)DA | 15.0 |
| IRG-819 | 0.20 |
| | Solvent Extraction Processing |
| Extraction Method | Soxhlet |
| Solvents | DMK/hex |
| Solvent Ratio | 59:41 |
| Extraction Period (h) | 24 |
| Post Extraction Drying | Vacuum |
| Method | oven |
| Drying Temperature | 60 (72 hr) |
| (° C.) | |
| Gravimetric Residuals | 6,366 |
| (ppm) | |
| | Mechanical Properties |
| | (Extracted Dry Polymer) |
| E-Modulus (MPa) | 2.32 |
| Tensile Strength (MPa) | 2.72 |
| Elongation (mean) (%) | 140 |
| Elongation (max) (%) | 172 |

### REFERENCES

All documents mentioned in this specification are incorporated herein by reference in their entirety.
EP 1,792,923
U.S. Pat. No. 5,290,892
U.S. Pat. No. 5,403,901
U.S. Pat. No. 5,674,960
U.S. Pat. No. 5,693,095
U.S. Pat. No. 5,861,031
U.S. Pat. No. 5,922,821
U.S. Pat. No. 6,241,766
U.S. Pat. No. 6,271,281
U.S. Pat. No. 6,281,319
U.S. Pat. No. 6,326,448
U.S. Pat. No. 6,780,899
U.S. Pat. No. 6,852,793
U.S. Pat. No. 7,354,980
U.S. Pat. No. 7,789,509
U.S. Pat. No. 7,790,825
U.S. Pat. No. 8,362,177
US 2001/0003162
US 2011/0313518
WO 96/40303
WO 00/79312
WO 2006/063994
WO 2007/094665
WO 2010/113600
WO 2011/107728

1. A monomer for a polymerisable composition, the monomer having the formula (I): 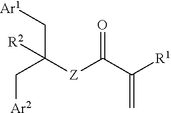
wherein:
R¹ is —H or alkyl;
—Z— is —O—, —NH—, or —N(R)—, where —R is optionally substituted alkyl or aryl;
-Ar¹ and -Ar² are each independently optionally substituted aryl; and
—R² is H, or, optionally substituted alkyl or aryl.

2. The monomer of claim 1, wherein —Z— is —O—.

3. (canceled)

4. The monomer of claim 1, wherein R¹ is —H or -Me.

5. (canceled)

6. The monomer of claim 1, wherein —R² is —H or optionally substituted alkyl.

7. The monomer of claim 1, wherein —R² is —H or -Me.

8. (canceled)

9. The monomer of claim 1, wherein —Ar¹ and -Ar² are each independently optionally substituted C₆₋₁₀ carboaryl.

10.-11. (canceled)

12. The monomer of claim 1, wherein -Ar¹ and -Ar² are each independently phenyl optionally substituted by 1 to 5 substituents selected from the groups consisting of alkyl, alkoxy, haloalkyl, halo and —N(R′)₂, where each R′ is independently —H or alkyl.

13. (canceled)

14. The monomer of claim 1, wherein -Ar¹ and -Ar² are each independently phenyl optionally substituted by 1 to 5 alkyl substituents, or -Ar¹ and Ar² are each independently phenyl optionally substituted by 1 to 5 alkoxy substituents.

15.-18. (canceled)

19. The monomer of claim 9, wherein -Ar¹ and -Ar² are each independently phenyl optionally having 1 substituent.

20. A polymerizable composition comprising one or more monomers according to claim 1.

21. (canceled)

22. The polymerisable composition according to claim 20, wherein the composition comprises a first monomer, and the amount of first monomer in the composition is 20 to 75 wt % of the composition

23. The polymerisable composition according to claim 22, the composition further comprising a second monomer for polymerisation with the first monomer, wherein the second monomer has an (alkyl)acrylate group.

24. The polymerisable composition according to claim 23, wherein the second monomer is selected from the group consisting of methyl acrylate, ethyl acrylate, n-propyl acrylate, n-butyl acrylate, t-butyl acrylate, n-hexyl acrylate, cyclohexyl acrylate, ethoxethyl acrylate, methoxyethyl acrylate, methyl methacrylate, ethyl methacrylate, n-propyl methacrylate, n-butyl methacrylate, t-butyl acrylate, methacrylate, cyclohexyl methacrylate, ethoxyethyl methacrylate, methoxyethyl methacrylate, isobornyl methacrylate, isobornyl acrylate, 2 phenylethyl methacrylate, 2-phenylethyl acrylate, 1,4-diphenylbutan-2-yl acrylate, 1,4 diphenylbutan-2-yl methacrylate, 1,5-diphenylpentan-3-ylacrylate, 1,5-diphenylpentan-3-yl methacrylate and mixtures thereof, or the second monomer is a monomer of formula (III): 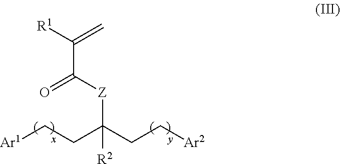
wherein:
—R¹ is —H or alkyl;
—Z is —O—, —NH or —NR—, where —R is optionally substituted alkyl or aryl;
-Ar¹ and -Ar² are each independently optionally substituted aryl;
—R² is —H, or optionally substituted alkyl or aryl; and
x and y are each independently 0 to 4, with the proviso that x and y are not both 0.

25.-27. (canceled)

28. The polymerisable composition according to claim 23, wherein the second monomer is present in the composition at 15 to 75 wt %.

29. The polymerisable composition according to claim 23, further comprising one or more third monomers for forming crosslinks with monomers in the polymerisable composition, wherein the third monomer has a plurality of (alkyl)acrylate groups or comprises a poly(oxyalklene) group.

30.-35. (canceled)

36. The polymerisable composition according to claim 22 further comprising one or more hydrophilic fourth monomers for polymerisation with the first monomer, wherein the fourth monomer has an (alkyl)acrylate group.

37.-42. (canceled)

43. The polymerisable composition according to claim 20 further comprising a thermally- or light-activated polymerisation initiator, a UV-light absorber, a blue light absorber, a tackiness modifying agent, or a combination thereof, wherein the UV light absorber and the blue-light absorber are optionally fixable.

44. The polymerisable composition according to claim 43 further comprising a UV-light absorber, which is optionally fixable, and the UV-light absorber is selected from the group consisting of 2-[3′-(2′H-benzotriazol-2′-yl)-4′-hydroxyphenyl]-ethylmethacrylate, 2-(4′-benzoyl-3′-hydroxyphenoxy)ethyl acrylate, 2-hydroxy-4-allyloxybenzophenone, 2-(2′-hydroxy-5-methacryloxyethylphenyl)-2H-benzotriazole, β-(4-benzotriazoyl-3-hydroxyphenoxy)-ethylacrylate, 4-(2-acryloxyethoxy)-2-hydroxybenzophenone, 4 methacryloyloxy-2-hydroxybenzophenone, 2-(2′-methacryloyloxy-5′-methylphenyl)benzotriazole, 2-(2′-hydroxy-5′-methacryloxyethylphenyl)-2H-benzotriazole, 2-[3′-tert-butyl-2′-hydroxy-5′-(3″-methacryloyloxypropyl)phenyl]-5-chlorobenzotriazole, 2-(3′-tert-butyl-5′-(3″-dimethylvinylsilylpropoxy)-2′-hydroxyphenyl]-5-methoxybenzotriazole, 2-(3′-allyl-2″-hydroxy-5′-methylphenyl)benzotriazole, 2-[3′-tert-butyl-2′-hydroxy-5′-(3″-methacryloyloxypropoxy)phenyl]-5-methoxybenzotriazole, 2-[3′-tert-butyl-2′-hydroxy-5-(3′-methacyloyloxypropoxy)phenyl]-5-chlorobenzotriazole, 2-(2′-hydroxy-5″-methacryloyloxyethylphenyl)-2H-benzotriazole and 2-(2′-hydroxy-3′-methallyl-5′-methylphenyl)benzotriazole.

45.-50. (canceled)

51. An ophthalmic lens formed from a polymer obtained or obtainable from a polymerisable composition according to claim 20.

52. The ophthalmic lens according to claim 51 which is an intraocular lens.

53-66. (canceled)
